(19) United States
(12) Patent Application Publication
(10) Pub. No.: US 2024/0428943 A1
(43) Pub. Date: Dec. 26, 2024

(54) EXPANDING THE DNA MISMATCH REPAIR DEFICIENCY/MICROSATELLITE INSTABILITY POPULATION THROUGH DIGITAL PATHOLOGY AND MUTATIONAL SIGNATURES

(71) Applicant: PathAI, Inc., Boston, MA (US)

(72) Inventors: Amaro N. Taylor-Weiner, Brooklyn, NY (US); Daniel Borders, New York, NY (US); Michael Drage, Wellesley, MA (US); Jake Conway, Lawrence, MA (US); Andrew H. Beck, Brookline, MA (US)

(73) Assignee: PathAI, Inc., Boston, MA (US)

(21) Appl. No.: 18/752,902

(22) Filed: Jun. 25, 2024

Related U.S. Application Data
(60) Provisional application No. 63/520,368, filed on Aug. 18, 2023.
(60) Provisional application No. 63/510,303, filed on Jun. 26, 2023.

(51) Int. Cl. G16H 50/20; G16H 20/17; G16H 30/00

(52) U.S. Cl. CPC G16H 50/20; G16H 20/17; G16H 30/00

(57) ABSTRACT

In some aspects, a method, system, and non-transitory computer-readable storage medium are described for predicting relevant biomarkers and patient response to immunotherapy treatment for one or more cancer types, including: using a first statistical model to determine cell-type and/or tissue-type characteristics associated with a pathology image of a patient; determining a plurality of human interpretable image features based on the cell-type and/or the tissue-type characteristics associated with the pathology image; using a second statistical model to classify the patient into one or more subpopulations of a plurality of subpopulations associated with a solid tumor based on the plurality of human interpretable image features; and predicting patient response to immunotherapy treatment based on the classification of the subpopulations. In some embodiments, a patient may be predicted to likely respond to immunotherapy treatment if the patient is classified into any of subpopulations MSS/dMMR, MSI, or MSS/TMB-H.

### CROSS-REFERENCE TO RELATED APPLICATIONS

This application claims the benefit under 35 U.S.C. § 119 (c) of U.S. Provisional Application Ser. No. 63/510,303, filed on Jun. 26, 2023, under Attorney Docket No. P1112.70024US00, entitled “EXPANDING THE DNA MISMATCH REPAIR DEFICIENCY/MICROSATELLITE INSTABILITY POPULATION THROUGH DIGITAL PATHOLOGY AND MUTATIONAL SIGNATURES,” and U.S. Provisional Application Ser. No. 63/520,368, filed on Aug. 18, 2023, under Attorney Docket No. P1112.70024US01, entitled “EXPANDING THE DNA MISMATCH REPAIR DEFICIENCY/MICROSATELLITE INSTABILITY POPULATION THROUGH DIGITAL PATHOLOGY AND MUTATIONAL SIGNATURES,” each of which is hereby incorporated herein by reference in its entirety.

### BACKGROUND

Microsatellite instability (MSI) is a biomarker for use of immunotherapy in solid tumors. Although DNA mismatch repair deficiency (dMMR) can cause MSI, dMMR itself is not traditionally considered a biomarker in absence of MSI positivity status. Several immunotherapy clinical trials have reported response to immunotherapy in MSS (microsatellite stable) patients.

### SUMMARY

The present disclosure relates to techniques for predicting biomarkers associated with a patient's response to cancer treatment such as immunotherapy treatment for solid tumors. The techniques provide a system, computerized method and non-transitory computer readable medium containing instructions for predicting biomarkers associated with a patient's response to immunotherapy treatment for solid tumors based on human interpretable image features (HIFs) extracted from a whole-slide pathology image(s) or pathology image patches of the patient and classifying the patient into one or more subpopulations of a plurality of subpopulations associated with a solid tumor. In some embodiments, the method includes: using a first statistical model to determine one or more cell-type labels and/or one or more tissue-type segmentations associated with a pathology image of a patient; determining a plurality of human interpretable image features based on the one or more cell-type labels and/or the one or more tissue-type segmentations associated with the pathology image; and using a second statistical model to classify the patient into one or more subpopulations of a plurality of subpopulations associated with a solid tumor based on the plurality of human interpretable image features.

In some embodiments, the plurality of subpopulations may include at least a first subpopulation having MSS/dMMR. The method may classify the patient into MSS/dMMR subpopulation based on the plurality of human interpretable image features. Subsequently, the method may predict whether the patient will likely respond to immunotherapy treatment for the solid tumor in response to determining that the patient is classified into MSS/dMMR subpopulation. In some embodiments, the plurality of subpopulations may further include other subpopulations including MSI, MSS/TMB-H (MSS patients having high tumor mutational burden), and MSS (for patients absent MSI, dMMR and TMB-H). The method may additionally predict that the patient will likely respond to immunotherapy treatment for the solid tumor in response to classifying the patient into any of the subpopulations MSI or MSS/TMB-H.

In some aspects, the techniques provide a system, computerized method and non-transitory computer readable medium containing instructions for predicting a patient's biomarkers associated with response to immunotherapy treatment for solid tumors based on classifying the patient directly from the pathology image(s) of the patient into one or more subpopulations of a plurality of subpopulations associated with a solid tumor. In some embodiments, the method includes: receiving one or more pathology images of a patient; using a statistical model and the one or more pathology images as input to the statistical model to classify the patient into one or more subpopulations of a plurality of subpopulations associated with a solid tumor, where the plurality of subpopulations comprises at least a first subpopulation having MSS/dMMR; in response to classification of the patient, predicting whether the patient will likely respond to immunotherapy treatment for the solid tumor. For example, the method may predict that the patient will respond to immunotherapy treatment in response to classifying the patient into subpopulation having MSS/dMMR. Alternatively, and/or additionally, the plurality of subpopulations may further include other subpopulations including MSI, MSS/TMB-H, and MSS (for patients absent MSI, dMMR and TMB-H). The method may additionally predict that the patient will likely respond to immunotherapy treatment for the solid tumor in response to classifying the patient into any of the subpopulations MSI or MSS/TMB-H.

In some aspects, the techniques provide a system, computerized method and non-transitory computer readable medium containing instructions for predicting biomarkers associated with a patient's response to immunotherapy treatment for solid tumors based on human interpretable image features (HIFs) extracted from the pathology image(s) of the patient. In some embodiments, the method includes: using a first statistical model to determine one or more cell-type labels and/or one or more tissue-type segmentations associated with a pathology image of a patient; determining a plurality of human interpretable image features based on the one or more cell-type labels and/or the one or more tissue-type segmentations associated with the pathology image; and using a second statistical model to predict whether the patient will likely respond to immunotherapy treatment based on the human interpretable image features.

The various techniques described above and further herein may be applied to predicting relevant biomarkers and patient response to immunotherapy treatment for any suitable solid tumors, such as colorectal cancer, endometrial cancer, gastric cancer, or other suitable type of cancers. While some embodiments described herein are described with respect to prediction of relevant biomarkers and patient response to treatment for particular cancer types, these embodiments may be equally suitable for any suitable cancer types.

Still other aspects, embodiments, and advantages of these exemplary aspects and embodiments, are discussed in detail below. Any embodiment disclosed herein may be combined with any other embodiment in any manner consistent with at least one of the objects, aims, and needs disclosed herein, and references to “an embodiment,” “some embodiments,” “an alternate embodiment,” “various embodiments,” “one embodiment” or the like are not necessarily mutually exclusive and are intended to indicate that a particular feature, structure, or characteristic described in connection with the embodiment may be included in at least one embodiment. The appearances of such terms herein are not necessarily all referring to the same embodiment. The accompanying drawings are included to provide illustration and a further understanding of the various aspects and embodiments and are incorporated in and constitute a part of this specification. The drawings, together with the remainder of the specification, serve to explain principles and operations of the described and claimed aspects and embodiments.

### BRIEF DESCRIPTION OF THE DRAWINGS

Various aspects of at least one embodiment are discussed below with reference to the accompanying figures, which are not intended to be drawn to scale. Where technical features in the figures, detailed description or any claim are followed by reference signs, the reference signs have been included for the sole purpose of increasing the intelligibility of the figures, detailed description, and claims. Accordingly, neither the reference signs nor their absence is intended to have any limiting effect on the scope of any claim elements. For purposes of clarity, not every component may be labeled in every figure. The figures are provided for the purposes of illustration and explanation and are not intended as a definition of the limits of the systems and methods described herein. In the figures:

FIG. 1 shows flow diagrams of example processes for predicting whether a patient has relevant biomarker(s) and will likely respond to immunotherapy treatment for solid tumors and training statistical models for the prediction, in accordance with some embodiments of the technology described herein.

FIG. 2 shows a flow diagram of an example process for predicting whether a patient has relevant biomarker(s) and will likely respond to immunotherapy treatment for solid tumors using various subsets of human interpretable image features, in accordance with some embodiments of the technology described herein.

FIG. 3 shows a variation of FIG. 1, where pathology images are used to directly classify a patient to subpopulations without determining human interpretable features as in example processes shown in FIG. 1, in accordance with some embodiments of the technology described herein.

FIG. 4 shows a variation of FIG. 1, where human interpretable features are used to train a statistical model to directly predict whether a patient will likely respond to immunotherapy treatment without classifying the patient to subpopulations as in example processes shown in FIG. 1, in accordance with some embodiments of the technology described herein.

FIG. 5A shows aspects of a pipeline overview for a system for quantifying tumor microenvironment (TME) and predicting molecular phenotypes or patient response to immunotherapy treatment for solid tumors, in accordance with some embodiments of the technology described herein.

FIGS. 5B and 5C show aspects of a dataset for use with the system in FIG. 5A for predicting molecular phenotypes, in accordance with some embodiments of the technology described herein.

FIG. 6 shows aspects of a human interpretable image feature extraction workflow, in accordance with some embodiments of the technology described herein.

FIGS. 7A-7F show aspects of an overview of human interpretable image features, in accordance with some embodiments of the technology described herein.

FIGS. 8A-8B show aspects of human interpretable image feature differences across cancer types, in accordance with some embodiments of the technology described herein.

FIGS. 9A-9C-4 show aspects of validation of human interpretable image features against immune markers, in accordance with some embodiments of the technology described herein.

FIGS. 10A-1-10B-2 show aspects of human interpretable image feature-based prediction of molecular phenotypes, in accordance with some embodiments of the technology described herein.

FIG. 11 schematically shows layers of a convolutional neural network, in accordance with some embodiments of the technology described herein.

FIG. 12A shows the genomic subpopulations breakdown by tumor stage for colorectal cancer, in accordance with some embodiments of the technology described herein.

FIG. 12B shows the genomic subpopulations breakdown by tumor stage for endometrial cancer, in accordance with some embodiments of the technology described herein.

FIG. 13A-13B show the distributions of a positively associated HIF (FIG. 13A) and negatively associated HIF (FIG. 13B) across the four subpopulations for colorectal cancer, in accordance with some embodiments of the technology described herein.

FIG. 14A shows the AUROC curve for MSI subpopulation prediction in colorectal cancer patients, in accordance with some embodiments of the technology described herein.

FIG. 14B shows the AUROC curve for dMMR subpopulation prediction in colorectal cancer patients, in accordance with some embodiments of the technology described herein.

FIG. 15A shows uncorrected p-values between subpopulations MSS/TMB-H and MSS in endometrial cancer patients, in accordance with some embodiments of the technology described herein.

FIG. 15B shows uncorrected p-values between subpopulations MSS/dMMR and MSS in endometrial cancer patients, in accordance with some embodiments of the technology described herein.

FIG. 15C shows uncorrected p-values between subpopulations MSI and MSS in endometrial cancer patients, in accordance with some embodiments of the technology described herein.

FIG. 16A shows uncorrected p-values between subpopulations MSS/dMMR and MSS/TMB-H in endometrial cancer patients, in accordance with some embodiments of the technology described herein.

FIG. 16B shows uncorrected p-values between subpopulations MSS/dMMR and MSI in endometrial cancer patients, in accordance with some embodiments of the technology described herein.

FIG. 16C shows uncorrected p-values between subpopulations MSS/TMB-H and MSI in endometrial cancer patients, in accordance with some embodiments of the technology described herein.

FIG. 17 shows a block diagram of a computer system on which various embodiments of the technology described herein may be practiced.

FIG. 18 shows an example of an additive multiple instance learning (MIL) model, in accordance with some embodiments of the technology described herein.

### DETAILED DESCRIPTION

Microsatellite instability (MSI) is a biomarker for use of immunotherapy in solid tumors. Although DNA mismatch repair deficiency (dMMR) can cause MSI, dMMR itself is not considered a biomarker in absence of MSI positivity status. Several immunotherapy clinical trials have reported response to immunotherapy in MSS (microsatellite stable) patients, demonstrating there is a need to identify these MSS patients prior to treatment. Further, there is a need to determine to what extent the response to immunotherapy in MSI patients is driven by molecular cancer cell intrinsic properties (e.g., MSI) versus extrinsic factors such as tumor microenvironment (TME) composition.

Existing systems may fall short on overcoming the current challenges and meeting the above mentioned needs. For example, existing methods for dMMR identification are restricted to whole-exome and whole-genome sequencing data which are not widely available or ordered in clinical practice. In digital pathology, existing methods predict MSI status derived from IHC (immunohistochemistry), PCR (polymerase chain reaction), or DNA-sequencing methods that count events in microsatellite regions, while ignoring the MSS patient population. Further, current digital pathology technologies or mutational signature methods applied to DNA-sequencing data are not able to evaluate the full spectrum of dMMR/MSI, especially as it requires quantifying the associated TME. These limitations prevent biomarker expansion to the subset of patients that are MSS but exhibit dMMR/MSI-like phenotypes or TMEs.

Accordingly, the inventors have developed techniques for integrating digital pathology with mutational signature analysis to detect dMMR in MSS patients, and detect MSI (as detected with IHC, PCR, microsatellite slippage quantification).

In various embodiments, univariate human interpretable features (HIF) approaches are used to identify TME features associated with an MSI/dMMR-like phenotype, most notably in MSS patients, and link it back to the underlying molecular subtype.

In some embodiments, both multivariable modeling using HIFs and additive multiple instance learning (aMIL) models deployed directly on whole-slide images (WSI) are able to infer dMMR status without the need for whole-exome or whole-genome sequencing.

In some embodiments, a digital pathology approach is described that can enable the investigation into what extent the response to immunotherapy in MSI patients is driven by molecular cancer cell intrinsic properties (e.g., MSI) versus extrinsic factors such as TME composition across the full spectrum of dMMR (e.g., in addition to merely MSI status). This is in contrast to previous digital pathology approaches for understanding dMMR, which were restricted to just the MSI population.

In some embodiments, integration of digital pathology with mutational signatures is described which may enable the expansion of the dMMR/MSI patient population that can receive immunotherapy.

The various embodiments described herein provide advantages over conventional systems. For example, human interpretable features (HIFs) and additive multiple instance learning (aMIL) models may be used to predict, and identify whole slide image (WSI)-derived features associated with, MSI positivity status and evidence of dMMR in microsatellite stable (MSS) tumors. Histopathology based dMMR/MSI may identify more patients who would benefit from immunotherapy, as opposed to MSI positive only patients in existing methods.

Various embodiments described herein are related to the associations with dMMR, and multivariable modeling of dMMR. In some exemplary implementations, the results show that integration of HIFs with MSI status, dMMR mutational signatures, and tumor mutational burden (TMB; another biomarker for immunotherapy) revealed a stepwise pattern for MSI-associated features, where MSS tumors with dMMR exhibited an intermediate phenotype. Multivariable modeling using the associated features enable prediction of MSI status in the population overall, and dMMR status in MSS subset of patients. Further, the TMEs of MSS tumors with dMMR are similar to the TMEs of MSI and TMB-H patients, which are two FDA-approved biomarkers for immunotherapy. MSS tumors with dMMR are associated with several MSI-related TME features when compared to MSS tumors without dMMR.

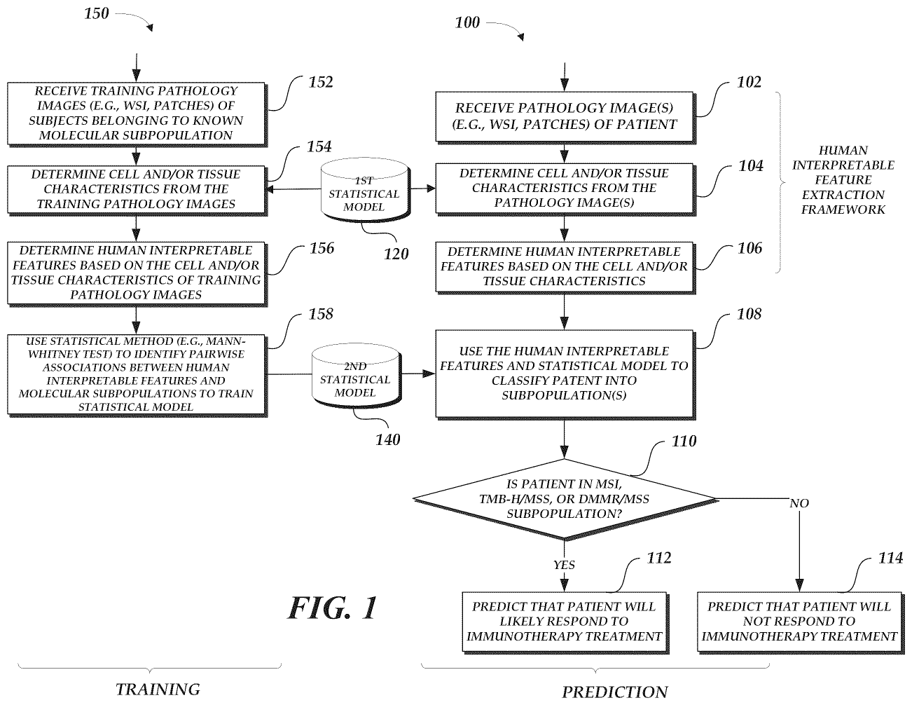

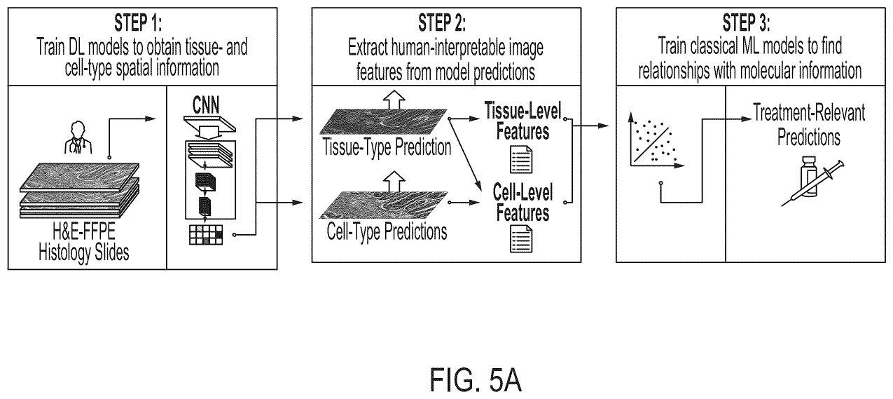

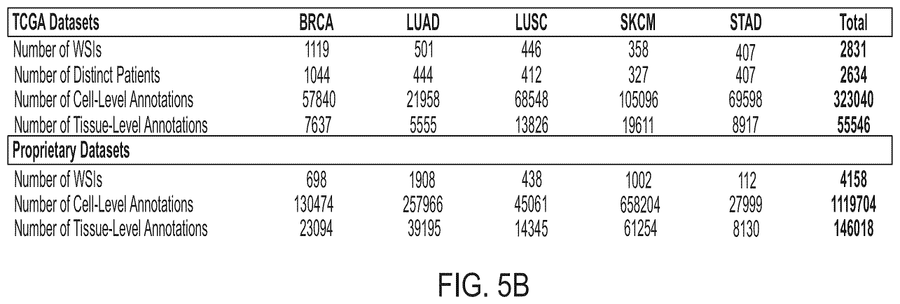

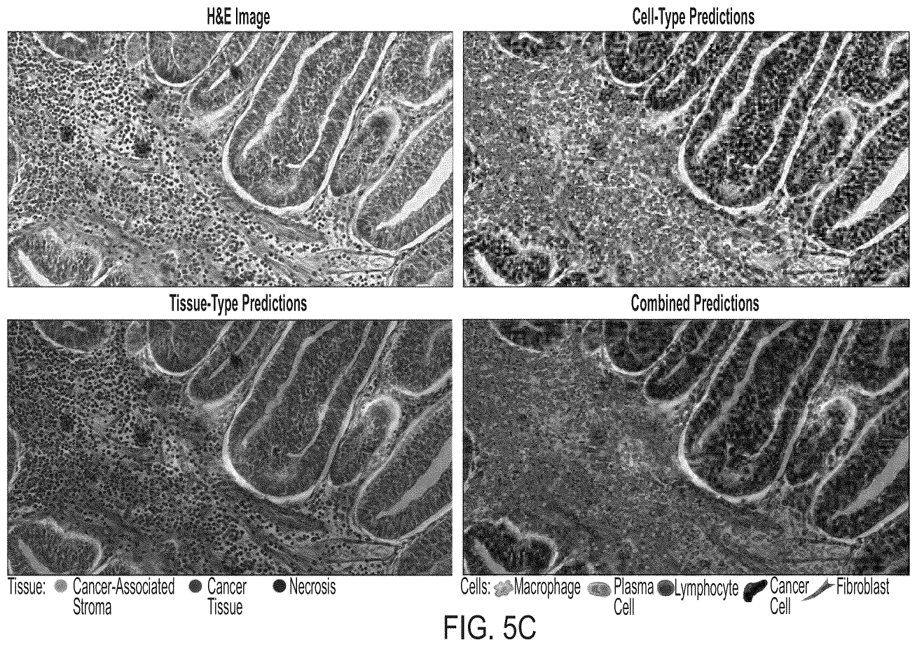

FIG. 1 shows flow diagrams of example processes for predicting whether a patient has relevant biomarker(s) and will likely respond to immunotherapy treatment for solid tumor and training statistical models for the prediction, in accordance with some embodiments of the technology described herein. In some embodiments, process 100 is provided that predicts whether a patient will likely respond to immunotherapy treatment for solid tumors. Process 100 may include receiving pathology image(s) of a patient at act 102; using a first statistical model (e.g., statistical model 120) to determine one or more cell-type labels and/or one or more tissue-type segmentations associated with a pathology image(s) of the patient, at act 104; and determining a plurality of human interpretable image features based on the one or more cell-type labels and/or the one or more tissue-type segmentations associated with the pathology image, at act 106. Acts 104, 106 may be implemented in a human interpretable feature extraction framework for quantifying tumor microenvironment and predicting molecular phenotypes or patient response to immunotherapy treatment for solid tumors. The human interpretable feature extraction framework will be described in detail further herein with reference to FIGS. 5A-11.

With further reference to FIG. 1, process 100 may further include using a second statistical model to classify the patient into one or more subpopulations of a plurality of subpopulations associated with a solid tumor based on the plurality of human interpretable image features, at act 108. In response to the classification of the patient, process 100 may further predict whether the patient will respond to immunotherapy treatment, at act 110.

The inventors have appreciated and acknowledged that there are certain biological relationships between a patient's genomic features. For example, dMMR may cause MSI, which is traditionally considered as a biomarker that responds to immunotherapy treatment. Further, dMMR may also cause TMB-H (e.g., patients having high tumor mutational burden (TMB), such as at least 10 mutations per megabase) because when the mismatch repair machinery in cells is not working properly, it may lead to more mutations (e.g., TMB-H). Conversely, high TMB (e.g., TMB-H) may cause dMMR because the higher TMB (e.g., more mutations a tumor accumulates results), the higher the probability of acquiring a mutation in a mismatch repair gene (and if mutated, may cause dMMR). As such, the presence of dMMR and TMB-H in MSS a patient may indicate that the patient may likely respond to immunotherapy treatment due to the probability that dMMR and/or TMB-H may cause MSI.

Accordingly, a patient may be classified into one or more of a plurality of subpopulations, where the classification of the patient may be used to predict whether the patient will respond to immunotherapy treatment. In some embodiments, a patient can belong to multiple subpopulations. For example, a MSI patient may typically have MSI, dMMR, and TMB-H (referred to as MSI subpopulation). A patient may have MSS without presence of dMMR or TMB-H (referred to as MSS subpopulation). These two subpopulations MSI and MSS may be considered as mutually exclusive and non-overlapping, whereas a patient can be determined to be MSI or MSS solely based on whether or not the patient is positive for MSI. Alternatively, a patient may be classified as MSS/dMMR or MSS/TMB-H, as further described in detail herein.

In some embodiments, a MSI subpopulation may include patients having MSI/dMMR/TMB-H, MSI/dMMR, or MSI/TMB-H. A MSS/dMMR subpopulation may include patients having MSS/dMMR or MSS/dMMR/TMB-H. An MSS/TMB-H subpopulation may include patients having MSS/TMB-H. A MSS subpopulation may include patients that are negative for MSI, dMMR or TMB-H.

In some embodiments, the classifications stated above and further herein may affect immunotherapy treatment decisions. For example, patients having MSI and TMB-H (irrespective of MSI status) may be predicted to respond to immunotherapy treatment, and thus, can be administered with immunotherapy treatment. In some embodiments, the immunotherapy treatment eligibility may also extend to MSS/dMMR patients, as similar to MSI and MSS/TMB-high patients. The techniques for predicting MSS/dMMR are further described in detail in the present disclosure.

In some embodiments, the plurality of subpopulations may include a first subpopulation having MSS/dMMR, a second subpopulation having MSI, and a third subpopulation having MSS/TMB-H. In some embodiments, the subpopulations may also include a fourth subpopulation in which the patient(s) do not have any of MSI, MSS/dMMR, and MSS/TMB-H. In the fourth subpopulation, the patient does not have MSI, MSS/dMMR or MSS/TMB-H, which are treated as biomarkers for immunotherapy in solid tumors.

Accordingly, act 110 may include determining whether a patient is classified into MSI using various embodiments described herein. Alternatively and/or additionally, MSI status may be determined by confirmative testing such as PCT. Responsive to determining that the patient is classified into MSI, process 100 may proceed to act 112 to predict that the patient may likely respond to immunotherapy treatment; otherwise act 110 may determine whether the patient is classified into MSS/dMMR using various embodiments described herein. Responsive to determining that the patient is classified into MSS/dMM, process 100 may proceed to act 112 to predict that the patient may likely respond to immunotherapy treatment. Responsive to determining that the patient is classified into the fourth subpopulation (e.g., when patient does not have MSI, MSS/dMMR or MSS/TMB-H), process 100 may proceed to act 114 to predict that the patient will not respond to immunotherapy treatment.

Although four subpopulations are described herein, it is appreciated that variations of subpopulations may also be possible. For example, in some embodiments, the plurality of subpopulations may include at least a subpopulation having MSS/dMMR. At act 110, process 100 may determine whether the patient is classified into the first subpopulation having MSS/dMMR. In response to determining that the patient is classified into the first subpopulation, process 100 may predict that the patient will likely respond to immunotherapy treatment, at act 112.

Now, process 100 is further described with further reference to FIG. 1. In some embodiments, act 104-108 may be performed based on whole-slide images or patches of whole-slide images. For example, act 102 may include receiving pathology image(s) in whole-slide images (e.g., from imaging scanning equipment) or pathology image patches that were previously generated from whole-slide images and stored. Alternatively, and/or additionally, act 102 may include generating the image patches (e.g., automatically, or semi-automatically, or manually via a user interface) from whole-slide images.

In some embodiments, act 104 may be trained to determine different cell and/or tissue characteristics for different cancer types. These cell and/or tissue characteristics may be used to subsequently determine human interpretable features for a given cancer type. Table 1 lists non-limiting examples of cell characteristics and tissue characteristics for different types of cancers: colorectal cancer, endometrial cancer, and gastric cancer. It is appreciated that the different groups of cell and/or tissue characteristics may be overlapping with common cell and/or tissue characteristics. For example, tissue characteristics cancer epithelium, cancer stroma, and necrosis may be used for all of the colorectal cancer, endometrial cancer, and gastric cancer. In other examples, cell characteristics cancer cell, fibroblast, macrophage, lymphocyte, plasma cell may be used for all of the three types of cancers.

TABLE 1
Tissue and cell characteristics per cancer type

| Cancer type | Tissue characteristics | Cell characteristics |
|---|---|---|
| Colorectal | Cancer epithelium, cancer stroma, | Cancer cell, fibroblast, macrophage, |
| Cancer | cancer gland lumen, buds and | lymphocyte, plasma cell |
| | poorly differentiated clusters, | |
| | mucin, necrosis | |
| Endometrial | Cancer epithelium, cancer stroma, | Cancer cell, fibroblast, macrophage, |
| Cancer | necrosis, normal | lymphocyte, plasma cell, endothelial |
| | | cell, neutrophil, eosinophil, other |
| Gastric Cancer | Cancer epithelium, cancer stroma, | Cancer cell, fibroblast, macrophage, |
| | mucin, necrosis | lymphocyte, plasma cell, neutrophil, |
| | | eosinophil, other stromal cells, other |

With further reference to FIG. 1, acts 104-114 may perform in a similar manner for predicting patient biomarkers and response to immunotherapy treatment for different cancer types, except that that the first statistical model for determining cell and/or tissue characteristics (e.g., statistical model 120) may be trained to extract different cell and/or tissue characteristics for different types of cancers, as shown in Table 1. In some embodiments, the statistical model 120 used for determining the cell and/or tissue characteristics at 104 may include a neural network, such as a convolutional neural network. It is appreciated that any other suitable machine learning models may be possible.

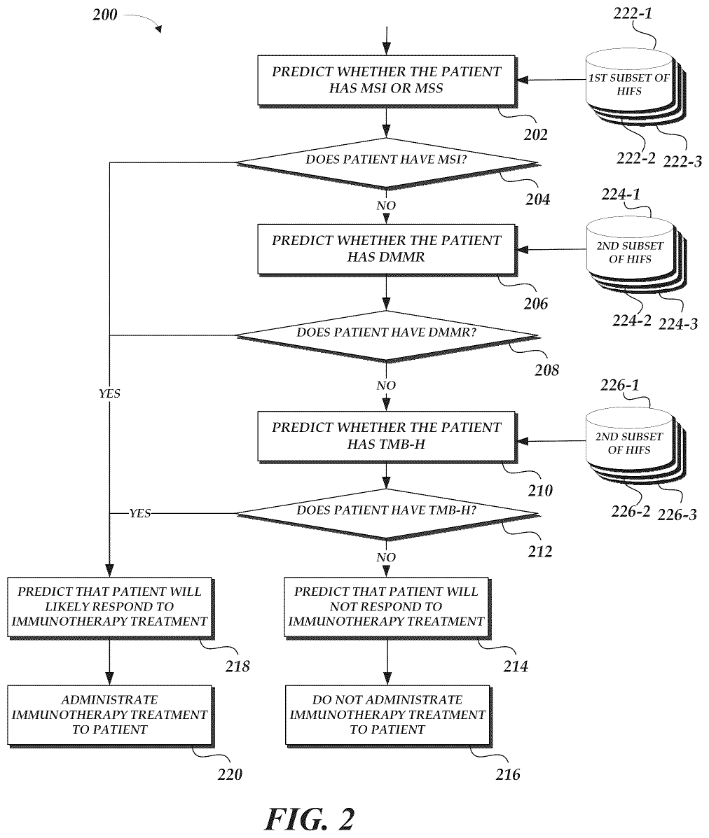

In some embodiments, the second statistical model (e.g., statistical model 140) may be trained to classify a patient into subpopulations for different types of cancers using different subsets of human interpretable image features, as will be further described in FIG. 2. Thus, different associations between human interpretable image features and the plurality of subpopulations may be established for different types of cancers in a training process (e.g., process 150). In some embodiments, statistical model 140 may include a machine learning classification model, e.g., multivariable logistic regression model. In some embodiments, the hyperparameters of multivariable logistic regression models for different cancer types may be different as the hyperparameters may be regularized to prevent overfitting, depending on the human interpretable features being used. It is appreciated that any other suitable machine learning classification models may be possible.

With further reference to FIG. 1, training process 150 may be performed to train the first statistical model for determining cell and/or tissue characteristics from pathology image(s) (e.g., statistical model 120) and the second statistical model for classifying a patient into subpopulations (e.g., statistical model 140), where the first and second statistical models (e.g., statistical models 120, 140) may be used in process 100 to predict patient biomarkers and response to immunotherapy treatment.

In some embodiments, training process 150 may include receiving training pathology images of training subjects belonging to known molecular subpopulations, at act 152; using the first statistical model to determine cell and/or tissue characteristics from the plurality of training pathology images, at act 154; determining one or more human interpretable image features based on the cell and/or tissue characteristics of the plurality of training pathology images, at act 156; and identifying a pairwise association between the one or more human interpretable image features and a subpopulation of the plurality of subpopulations, at act 158. Thus, the second statistical model (e.g., statistical model 140) may include pairwise associations between the human interpretable image features and the plurality of subpopulations. As described above and further herein, in some embodiments, the statistical model 140 may include a logistic regression model, in which these pairwise associations between the human interpretable image features and the plurality of subpopulations may be used to predict one or more subpopulations for a new patient (e.g., in act 108).

In training, act 152 may include receiving different training pathology images for different types of cancer, where each training pathology image may pertain to a specific type of cancer. Act 154 may be performed in a similar manner as act 104 is performed, such as using the same statistical model (e.g., statistical model 120) and same cell and/or tissue characteristics as those determined in act 104, depending on the type of cancer. In other words, different cell and/or tissue characteristics (e.g., those shown in Table 1) may be extracted from training samples/images/patches for different types of cancers. Similarly, act 156 may be performed in a similar manner as act 106. For example, similar human interpretable image features as those determined in act 106 may be extracted from the training pathology images during the training. In other words, different human interpretable image features may be extracted from training pathology images for different types of cancers. Examples of these different human interpretable image features are further described herein with reference to FIG. 2.

With further reference to FIG. 1, in training, the training pathology images may include H&E-stained whole slide images (or image patches, which may be provided for training or generated from the training whole slide images). Statistical model 120 (e.g., a convolutional neural network model) may be trained using the training pathology images to extract cell and/or tissue characteristics, and subsequently human interpretable features may be extracted based on the cell and/or tissue characteristics. Ground-truth MSI status (classification) for the training subjects may be determined using existing methods, such as polymerase chain reaction (PCR), which generates a binary result (e.g., MSI or not MSI). In some embodiments, MSI may also be classified by other clinically validated and approved assays, such as IHC of the associated genes MSH2, MSH6, MLH2, and PSM2 (in which only one gene needs to be positive for MSI).

Ground-truth TMB-H status (classification) for the training subjects may be determined from whole-exome sequencing mutation calls. For example, TMB-H may be determined by counting the number of mutations in the tumor divided by the length of the genome that was sequenced in megabase units. This determines mutations per megabase, which is a standard unit for reporting TMB. Clinically, a TMB threshold of 10 mutations per megabase may be used to classify a tumor as TMB-H, which is a biomarker for immunotherapy. In some embodiments, TMB-H may also be classified from whole-exome and whole-genome sequencing, as well as clinically validated and approved assays, such as panel sequencing. Panel sequencing only sequences relevant genes (usually ˜500-700 genes associated with cancer) and may be cheaper, faster (turnaround time), and easier to be interpreted (due to genes having known associations, functions, and applicable drugs).

The presence of dMMR for a training subject may be determined by mutational signature analysis. In some embodiments, mutational signature analysis may include whole-exome sequencing (e.g., sequencing the part of the genome that codes for genes) or whole-genome sequencing (e.g., sequencing the entire genome), which can be exclusively academic pursuits and rare in clinical settings. In some embodiments, mutational signature analysis may be performed without the whole-exome sequencing or whole-genome sequencing. Instead, dMMR may be identified based on the pattern of mutations in the tumor. Additionally, whereas the results from mutational signature analysis may be treated as a binary feature (presence vs. absence of the signature), the presence of dMMR may be determined when a number of mutations attributed to the signature exceeds a threshold, to prevent false positives. For example, when there are at least 30 mutations attributed to the signature, which is approximately 1 mutation per megabase for whole-exome sequencing, dMMR presence may be determined. In some embodiments, the presence of dMMR may be determined by identifying signatures 6, 15, 20, and 26 via the deconstructSigs mutational signature R package (see Rosenthal R, McGranahan N, Herrero J, Taylor B S, Swanton C. DeconstructSigs: delineating mutational processes in single tumors distinguishes DNA repair deficiencies and patterns of carcinoma evolution. Genome Biol. 2016 Feb. 22; 17:31. Doi: 10.1186/s13059-016-0893-4. PMID: 26899170; PMCID: PMC4762164), which is incorporated herein by reference.

Returning to FIG. 1, based on the MSI (or MSS, which is MSI negative), dMMR, and TMB-H statuses as described above and further herein, training subjects may be classified into subpopulations. In some embodiments, because a combination of MSI/MSS, dMMR, or TMB-H may be detected, these combinations may correspond to one of the four subpopulations. In other words, a training subject may be classified into one of the four subpopulations: MSI, MSS/dMMR, MSS/TMB-H, and MSS depending on the combination of presence/absence of genomic markers: MSI (or MSS), dMMR, and TMB-H. Table 2 lists an example of how a combination of genomic markers is classified into a subpopulation.

TABLE 2
Presence of genomic markers and classification of subpopulation

| | Classification of |
|---|---|
| Genomic markers detected | subpopulation |
| MSI/dMMR/TMB-H | MSI |
| MSI/dMMR | MSI |
| MSI/TMB-H | MSI |
| MSI | MSI |
| MSS/dMMR/TMB-H | MSS/dMMR |
| MSS/dMMR | MSS/dMMR |
| MSS/TMB-H | MSS/TMB-H |
| MSS (no presence of MSI, dMMR or TMB-H) | MSS |

Subsequently, pairwise associations between human interpretable features and molecular subpopulations may be established for the training subjects based on the human interpretable features extracted from the training pathology images of the training subjects and detected genomic markers of the training subjects. For example, Mann-Whitney tests may be used to identify the pairwise associations. It is appreciated that other tests may be performed to identify the pairwise associations between human interpretable features and molecular subpopulations.

FIG. 2 shows a flow diagram of an example process 200 for predicting whether a patient has relevant biomarker(s) and will likely respond to immunotherapy treatment for solid tumors using various subsets of human interpretable features, in accordance with some embodiments of the technology described herein. In some embodiments, process 200 may implement one or more acts in process 100. For example, process 200 may implement acts 108, 110, 112, 114 (in FIG. 1) to predict whether a patient will likely respond to immunotherapy treatment as will be further described.

With further reference to FIG. 2, process 200 may include predicting whether a patient has MSI or MSS, at act 202. In some embodiments, act 202 may use a first portion of the second statistical model (e.g., statistical model 140 in FIG. 1) to predict whether the patient has MSI or MSS, where the first portion of the second statistical model may be associated with a first subset of the plurality of human interpretable image features (e.g., 222). As shown in FIG. 2, the first subset of the plurality of human interpretable image features may be different (e.g., shown as 222-1, 222-2, 222-3, . . . ) for different types of cancers. For example, Table 3 lists examples of human interpretable image features for classifying between MSI and MSS for endometrial cancer. Subsequently, process 200 may include determining whether the patient has MSI at act 204. In response to predicting that the patient has MSI, process 200 may proceed to act 218 and predict that the patient will likely respond to immunotherapy treatment.

TABLE 3
Human interpretable image features for classifying
between MSI and MSS subpopulations

count proportion of lymphocyte cells over all predicted cells in cancer
stroma
density of lymphocyte cells in cancer stroma
density of immune cells in cancer stroma
density of plasma cell cells in cancer stroma
density of lymphocyte cells in tumor
density of lymphocyte cells in esi_0120
density of lymphocyte cells in esi_040
density of immune cells in tissue
density of plasma cell cells in tumor
density of plasma cell cells in esi_0120
density of lymphocyte cells in tissue
count proportion of immune cells over all predicted cells in cancer stroma
density of plasma cell cells in esi_040
density of plasma cell cells in tissue
count proportion of lymphocyte cells over all predicted cells in esi_040
density of immune cells in tumor
density of immune cells in esi_0120
density of immune cells in esi_040
count proportion of lymphocyte cells over all predicted cells in esi_0120
count proportion of lymphocyte cells over immune cells in esi_0120
count proportion of lymphocyte cells over immune cells in tumor
count proportion of lymphocyte cells over all predicted cells in tumor
count proportion of lymphocyte cells over immune cells in esi_040
count proportion of lymphocyte cells over immune cells in cancer stroma
count proportion of plasma cell cells over all predicted cells in cancer
stroma
density of lymphocyte cells in cancer
count proportion of lymphocyte cells over immune cells in cancer
count proportion of lymphocyte cells over all predicted cells in tissue
count proportion of plasma cell cells over all predicted cells in esi_040
count proportion of plasma cell cells over all predicted cells in esi_0120
density of neutrophil cells in tissue
count proportion of plasma cell cells over all predicted cells in tumor
count prop endothelial cells over all predicted cells in cancer stroma
count proportion of immune cells over all predicted cells in esi_040
count proportion of immune cells over all predicted cells in tissue
count proportion of plasma cell cells over all predicted cells in tissue
count proportion of immune cells over all predicted cells in esi_0120
count proportion of lymphocyte cells over all predicted cells in cancer
density of plasma cell cells in cancer
count proportion of endothelial cells over all predicted cells in esi_0120
density of immune cells in cancer
count proportion of endothelial cells over all predicted cells in esi_040
count proportion of immune cells over all predicted cells in tumor
count proportion of neutrophil cells over immune cells in esi_0120
density of eosinophil cells in cancer stroma
count proportion of neutrophil cells over immune cells in cancer stroma
count proportion of neutrophil cells over immune cells in esi_040
count proportion of macrophage cells over immune cells in cancer stroma
density of macrophage cells in cancer stroma
count proportion of neutrophil cells over immune cells in tumor
density of eosinophil cells in tumor
density of macrophage cells in esi_040
count proportion of lymphocyte cells over immune cells in tissue
count proportion of plasma cell cells over all predicted cells in cancer
count proportion of macrophage cells over immune cells in esi_0120
density of macrophage cells in esi_0120
density of macrophage cells in tumor
density of cancer cells in cancer
count proportion of macrophage cells over immune cells in cancer
count proportion of endothelial cells over all predicted cells in tumor
density of eosinophil cells in esi_040

With further reference to FIG. 2, in response to predicting that the patient does not have MSI (at act 204), process 200 may proceed to act 206 and use a second portion of the second statistical model (e.g., statistical model 140 in FIG. 1) to predict whether the patient has dMMR among the subset of MSS patients, where the second portion of the second statistical model may be associated with a second subset of the plurality of human interpretable image features (e.g., 224). As shown in FIG. 2, the second subset of the plurality of human interpretable image features may be different (e.g., shown as 224-1, 224-2, 224-3, . . . ) for different types of cancers. For example, Table 4 lists examples of human interpretable image features for classifying between MSS/dMMR and MSS subpopulations for endometrial cancer. Subsequently, process 200 may include determining whether the patient has dMMR at act 208. In response to predicting that the patient has dMMR, process 200 may proceed to act 218 and predict that the patient will likely respond to immunotherapy treatment.

TABLE 4
Human interpretable image features for classifying
between MSS/dMMR and MSS subpopulations

count proportion of lymphocyte cells over all predicted cells in cancer
stroma
density of lymphocyte cells in cancer stroma
density of immune cells in cancer stroma
density of plasma cell cells in cancer stroma
density of lymphocyte cells in tumor
density of lymphocyte cells in esi_0120
density of lymphocyte cells in esi_040
density of plasma cell cells in tumor
density of plasma cell cells in esi_0120
density of lymphocyte cells in tissue
density of plasma cell cells in esi_040
density of plasma cell cells in tissue
count proportion of lymphocyte cells over all predicted cells in esi_040
count proportion of lymphocyte cells over all predicted cells in esi_0120
count proportion of lymphocyte cells over immune cells in esi_0120
count proportion of lymphocyte cells over immune cells in tumor
count proportion of lymphocyte cells over all predicted cells in tumor
count proportion of lymphocyte cells over immune cells in esi_040
count proportion of plasma cell cells over all predicted cells in cancer
stroma
count proportion of lymphocyte cells over immune cells in cancer
count proportion of lymphocyte cells over all predicted cells in tissue
count proportion of plasma cell cells over all predicted cells in esi_0120
count proportion of plasma cell cells over all predicted cells in tumor
count proportion of endothelial cells over all predicted cells in cancer
stroma
count proportion of plasma cell cells over all predicted cells in tissue
count proportion of endothelial cells over all predicted cells in esi_0120
count proportion of endothelial cells over all predicted cells in esi_040
count proportion of neutrophil cells over immune cells in esi_0120
count proportion of neutrophil cells over immune cells in cancer stroma
count proportion of neutrophil cells over immune cells in esi_040
count proportion of neutrophil cells over immune cells in tumor
count proportion of lymphocyte cells over immune cells in tissue
density of endothelial cells in cancer stroma
count proportion of neutrophil cells over immune cells in tissue

With further reference to FIG. 2, in response to predicting that the patient does not have dMMR (at act 208), process 200 may proceed to act 210 and use a third portion of the second statistical model (e.g., statistical model 140 in FIG. 1) to predict whether the patient has TMB-H, where the third portion of the second statistical model may be associated with a third subset of the plurality of human interpretable image features (e.g., 226). As shown in FIG. 2, the third subset of the plurality of human interpretable image features may be different (e.g., shown as 226-1, 226-2, 226-3, . . . ) for different types of cancers. For example, Table 5 lists examples of human interpretable image features for classifying between MSS/TMB-H and MSS subpopulations for endometrial cancer. Subsequently, process 200 may include determining whether the patient has TMB-H at act 212. In response to predicting that the patient has TMB-H, process 200 may proceed to act 218 and predict that the patient will likely respond to immunotherapy treatment.

TABLE 5
Human interpretable image features for classifying
between MSS/TMB-H and MSS subpopulations

density of immune cells in cancer stroma
density of plasma cell cells in cancer stroma
density of plasma cell cells in tumor
density of plasma cell cells in esi_0120
count proportion of immune cells over all predicted cells in cancer stroma
density of plasma cell cells in esi_040
count proportion of plasma cell cells over all predicted cells in cancer
stroma
density of neutrophil cells in tissue
count proportion of endothelial cells over all predicted cells in esi_0120
count proportion of endothelial cells over all predicted cells in esi_040
count proportion of plasma cell cells over immune cells in cancer stroma

With further reference to FIG. 2, in response to predicting that the patient does not have TMB-H (at act 212), process 200 may proceed to act 214 to predict that the patient will not respond to immunotherapy treatment. Additionally, and/or alternatively, in response to determining that the patient will likely respond to immunotherapy treatment (e.g., at act 218), process 200 may further proceed with administrating immunotherapy treatment to patient, at act 220. Conversely, in response to predicting that the patient will not respond to immunotherapy treatment (e.g., at act 214), process 200 may further proceed with not administrating immunotherapy treatment to patient, at act 216.

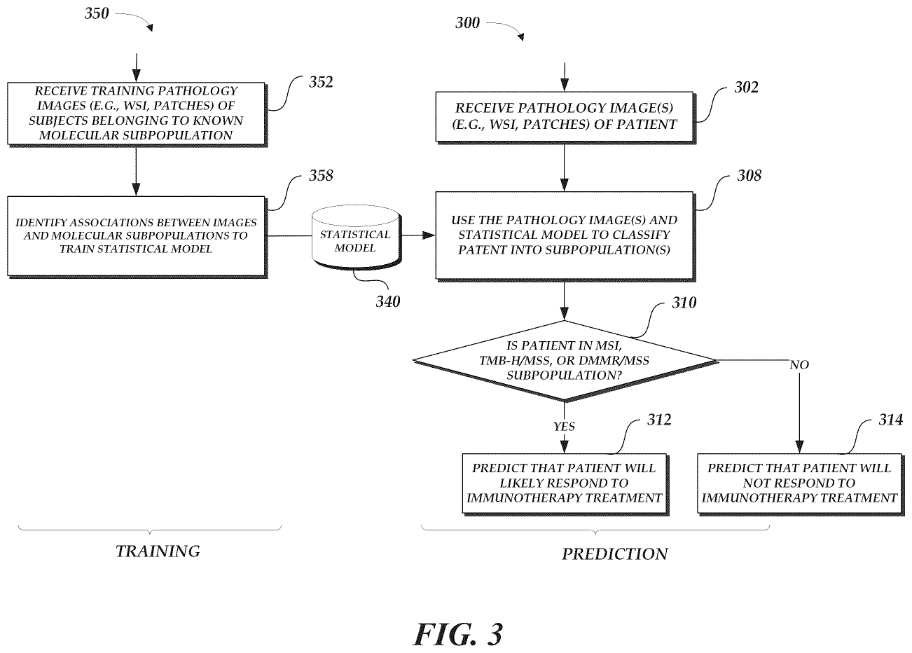

FIG. 3 shows a variation of FIG. 1, where pathology images are used to directly classify a patient to subpopulations without determining human interpretable features as in example processes shown in FIG. 1, in accordance with some embodiments of the technology described herein. In some embodiments, process 300 may be a variation of process 100 (in FIG. 1) with a difference being that statistical model 340 can be trained to directly classify a patient to subpopulations based on the pathology image(s) of the patient. In other words, acts 104 and 106 and corresponding training operations 154, 156 (in FIG. 1) are not needed. Accordingly, process 300 may include receiving one or more pathology images of a patient, at act 302; using a statistical model (e.g., 340) and the one or more pathology images as input to the statistical model to classify the patient into one or more subpopulations of a plurality of subpopulations associated with solid tumor, at act 308.

Similar to process 100, in some embodiments, the plurality of subpopulations may include a first subpopulation having MSS/dMMR, a second subpopulation having MSI, and a third subpopulation having MSS/TMB-H (MSS). In some embodiments, the subpopulations may also include a fourth subpopulation in which the patient(s) do not have any of MSI, MSS/dMMR, and MSS/TMB-H. In the fourth subpopulation, the patient does not have MSI, MSS/dMMR or MSS/TMB-H, which are treated as biomarkers for immunotherapy in solid tumors.

Accordingly, act 310 may include determining whether a patient is classified into MSI using various embodiments described herein. Alternatively and/or additionally, MSI status may be determined by confirmative testing such as PCT. Responsive to determining that the patient is classified into MSI, process 300 may proceed to act 312 to predict that the patient may likely respond to immunotherapy treatment; otherwise act 310 may determine whether the patient is classified into MSS/dMMR using various embodiments described herein. Responsive to determining that the patient is classified into MSS/dMM, process 300 may proceed to act 312 to predict that the patient may likely respond to immunotherapy treatment. Responsive to determining that the patient is classified into the fourth subpopulation (e.g., when patient does not have MSI, MSS/dMMR or MSS/TMB-H), process 300 may proceed to act 314 to predict that the patient will not respond to immunotherapy treatment.

Although four subpopulations are described herein, it is appreciated that variations of subpopulations may also be possible. For example, in some embodiments, the plurality of subpopulations may include at least a subpopulation having MSS/dMMR. At act 310, process 300 may determine whether the patient is classified into the first subpopulation having MSS/dMMR. In response to determining that the patient is classified into the first subpopulation, process 300 may predict that the patient will likely respond to immunotherapy treatment, at act 312.

In some embodiments, acts in FIG. 3 and FIG. 1 with reference numerals alike may be performed in similar manners. For example, the one or more pathology images of a patient received at act 302 may include whole-slide images or patches, similar to act 102. In training, acts 352 and 358 may be performed respectively similar to acts 152, 158 in FIG. 1. Similar to process 100 (in FIG. 1), process 300 may be configured to predict whether a patient will respond to immunotherapy treatment for various types of cancers, such as colorectal cancer, endometrial cancer, or gastric cancer. As such, statistical model (e.g., model 340) may be trained for different types of cancers using different sets of training pathology images (and respective ground truth data).

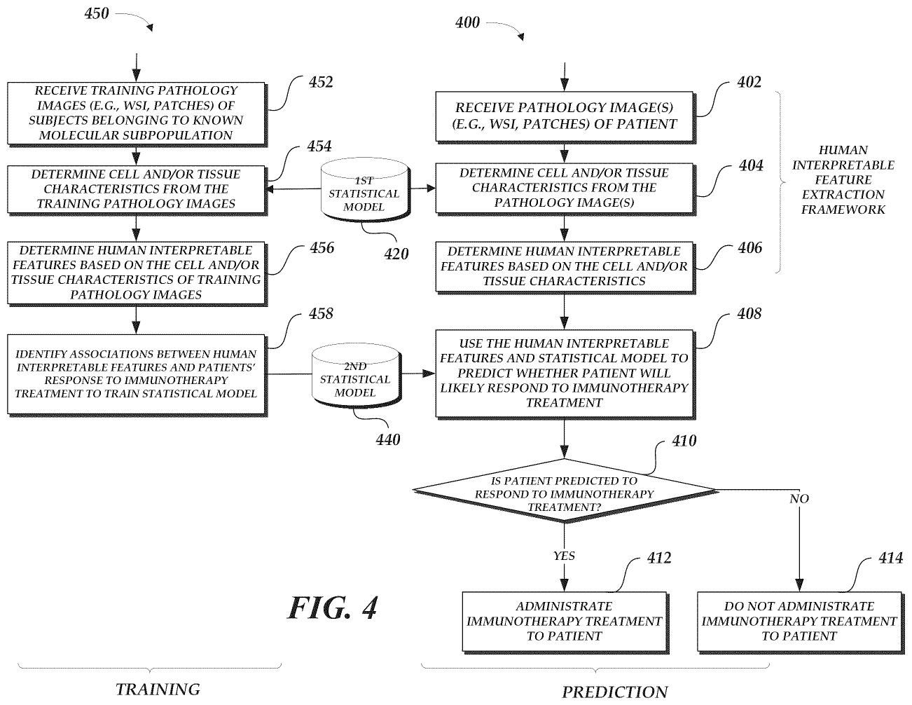

FIG. 4 shows a variation of FIG. 1, where human interpretable features are used to train a statistical model to directly predict whether a patient will respond to immunotherapy treatment without classifying a patient to subpopulations as in example processes shown in FIG. 1, in accordance with some embodiments of the technology described herein. In some embodiments, process 400 may be a variation of process 100 (in FIG. 1) with a difference being that statistical model 440 can be trained to directly predict whether or not the patient has relevant biomarkers and will respond to immunotherapy treatment, without classifying the patient into subpopulations. Similarly, in training, process 450 may include identifying associations between human interpretable features and patient response to immunotherapy treatment to train the statistical model, at act 458. Acts 452, 454 and 456 mirror acts 152, 154 and 165, respectively. Thus, the training data will include ground truth data as to whether each training subject responds to immunotherapy treatment. Contrary to process 100 (FIG. 1), process 450 does not need to determine the presence/absence of MSI, dMMR, or TMB-H associated with subpopulations. In other words, the biology captured by the human interpretable image features may drive immunotherapy response without considering the genomic markers (e.g., MSI, dMMR, TMB-H).

Accordingly, process 400 may include receiving one or more pathology images of a patient, at act 402; using a first statistical model (e.g., statistical model 420) to determine one or more cell-type labels and/or one or more tissue-type segmentations associated with the one or more pathology image(s) of the patient, at act 404; determining a plurality of human interpretable image features for a solid tumor based on the one or more cell-type labels and/or the one or more tissue-type segmentations associated with the pathology images, at act 406; and using a second statistical model (e.g., statistical model 440) and the plurality of human interpretable image features as input to the second statistical model to predict whether the patient will respond to immunotherapy treatment for the solid tumors, at act 408. With further reference to FIG. 4, process 400 may determine whether a patient is predicted to respond to immunotherapy treatment, at act 410. In response to determining that the patient is predicted to respond to immunotherapy treatment, process 400 may proceed to administrate immunotherapy treatment to the patient, at act 412; otherwise, process 400 may proceed to not administrate immunotherapy treatment to the patient, at act 414.

Similar to processes 100, 150 in FIG. 1, the first statistical model 420 may be trained for different types of cancers (e.g., colorectal cancer, endometrial cancer, or gastric cancer, or other suitable types of cancers), based on different training images (or patches) of patients having different types of cancers. Similar to FIG. 1, the statistical model 420 may also be trained to extract different sets of cell and/or tissue characteristics for different types of cancers. Similar to FIG. 1, statistical model 440 may also be trained for different types of cancers (e.g., colorectal cancer, endometrial cancer, or gastric cancer, or other suitable types of cancers), based on different sets of human interpretable features.

In some embodiments, the human interpretable features framework described in embodiments of FIGS. 1-4 are further described in detail with reference to FIGS. 5A-11. These embodiments in FIGS. 5A-11 may implement the human interpretable features framework in embodiments described in FIGS. 1-4. For example, the system shown in FIG. 5A may be implemented in processes 100 (FIG. 1) and 400 (FIG. 4).

The inventors have appreciated and acknowledged that end-to-end deep learning models that infer outputs directly from raw images present significant risks for clinical settings, including fragility of machine learning models to dataset shift, adversarial attack, and systematic biases present in training data. Many of these risks stem from the well-known problem of poor model interpretability. “Black-box” model predictions are difficult for users to interrogate and understand, leading to user distrust. Without reliable means for understanding when and how vulnerabilities may become failures, computational methods may face difficulty achieving widespread adoption in clinical settings.

To address issues with the conventional approaches, the inventors have developed solutions for automated computation of human interpretable image features (HIFs) to predict clinical outcomes. The described HIF-based prediction models may mirror the pathology workflow of searching for distinctive, stage-defining features under a microscope and offer opportunities for pathologists to validate intermediate steps and identify failure points. In addition, the described HIF-based solutions may enable incorporation of histological knowledge and expert pixel-level annotations which increases predictive power. Studied HIFs span a wide range of visual features, including stromal morphological structures, cell and nucleus morphologies, shapes and sizes of tumor regions, tissue textures, and the spatial distributions of tumor-infiltrating lymphocytes (TILs).

Further, the inventors have appreciated the increasingly clear relationship between the TME and patient response to targeted therapies, such as immunotherapy treatment for solid tumors. For instance, immuno-supportive phenotypes, which exhibit greater baseline antitumor immunity and improved immunotherapy response, have been linked to the presence of TILs and elevated expression of programmed death-ligand 1 (PD-L1) on tumor-associated immune cells. In contrast, immuno-suppressive phenotypes have been linked to the presence of tumor-associated macrophages and fibroblasts, as well as reduced PD-L1 expression. HIF-based approaches have the potential to provide an interpretable window into the composition and spatial architecture of the TME in a manner that is complementary to conventional genomic approaches.

The inventors have developed a computational pathology pipeline that can integrate high-resolution cell- and tissue-level information from WSIs to predict treatment-relevant, molecularly-derived phenotypes across different cancer types. In doing so, in a non-limiting example, the inventors introduce a diverse collection of HIFs ranging from simple cell (e.g. density of lymphocytes in cancer tissue) and tissue quantities (e.g. area of necrotic tissue) to complex spatial features capturing tissue architecture, tissue morphology, and cell-cell proximity. Notably, the inventors have demonstrated that such features can generalize across cancer types and provide a quantitative and interpretable link to specific and biologically-relevant characteristics of each TME.

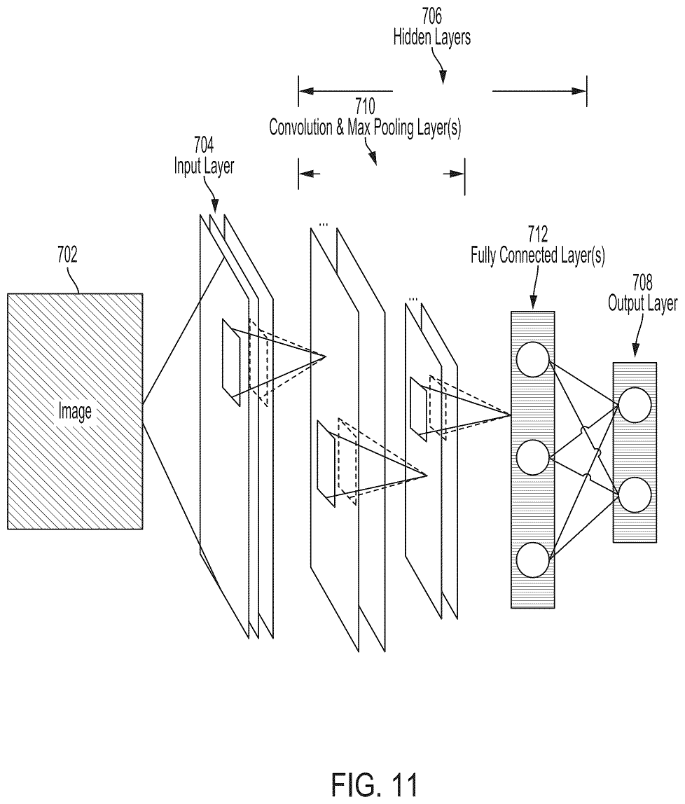

Throughout this disclosure, a convolutional neural network is used as an exemplary basis for a deep learning model that may be used in accordance with some embodiments. For example, a convolutional neural network may be used for statistical model 120 (FIG. 1), 420 (FIG. 4). However, it should be appreciated that other types of statistical models may alternatively be used, and embodiments are not limited in this respect. Other types of statistical models that may be used include a support vector machine, a neural network, a regression model, a random forest, a clustering model, a Bayesian network, reinforcement learning, metric learning, a genetic algorithm, or another suitable statistical model. More details for training the convolutional neural network are provided with respect to FIG. 11.

In some aspects, the described systems and methods provide for training and/or using one or more models to predict one or more molecular phenotypes based on human interpretable image features extracted from whole-slide images or other suitable images. The described systems and methods may be implemented on a computer system, such as the system discussed with respect to FIG. 25, or another suitable computer system, or a combination thereof.

### I. HUMAN INTERPRETABLE FEATURES FRAMEWORK

Further aspects of the technology may be understood based on the non-limiting illustrative embodiments described further below. Examples of specific implementations are provided herein for illustrative purposes only and are not intended to be limiting. While some embodiments described herein are described with respect to prediction of molecular phenotypes from whole-slide images and/or image patches, these embodiments may be equally suitable for other histopathology, histology, or pathology applications. While some embodiments described herein are described with respect to prediction of particular molecular phenotypes, these embodiments may be equally suitable for prediction of any molecular phenotype.

### Dataset Characteristics and Fully-Automated Pipeline Design

In some embodiments, in order to test the approach on a diverse array of histopathology images, 2,831 hematoxylin and eosin (H&E) stained, formalin-fixed and paraffin-embedded (FFPE) WSIs from the The Cancer Genome Atlas (TCGA), corresponding to 2,634 distinct patients, were obtained. (The Cancer Genome Atlas dataset is available at www.cancer.gov/about-nci/organization/ccg/research/structural-genomics/tega. The relevant data includes 2,831 hematoxylin and eosin-stained WSIs of breast cancer, non-small cell lung adenocarcinoma, non-small cell lung squamous cell carcinoma, gastric adenocarcinoma, and skin cutaneous melanoma specimens from 2,634 patients.) These images, each scanned at either 20× or 40× magnification, represented patients with skin cutaneous melanoma (SKCM), stomach adenocarcinoma (STAD), breast cancer (BRCA), lung adenocarcinoma (LUAD), and lung squamous cell carcinoma (LUSC) from 95 distinct clinical sites. To supplement the TCGA analysis cohort, 4,158 additional WSIs for the five cancer types were obtained to improve model robustness.

To maximize capture of this information, images (n=5) were excluded only if they failed basic quality control checks as determined by expert pathologists. Criteria for quality control were limited to mislabeling of tissue, excessive blur, or insufficient staining, but the described systems and methods are not so limited. For both TCGA and additional WSIs, cell- and tissue-level annotations were collected from a network of pathologists, amounting to >1.4 million cell-type point annotations and >200 thousand tissue-type region annotations.

The inventors used the resulting slides and annotations to design a fully automated pipeline to extract HIFs from these slides (summarized in FIG. 5A, illustrating a methodology for extracting HIFs from high-resolution, digitized H&E images). First, deep learning models were trained to label cells (“cell-type models”) and segment tissue regions (“tissue-type models”). Training and validation of models was conducted on a development set of 1,561 TCGA WSIs, supplemented by the 4,158 additional WSIs (n=5719). FIG. 5B illustrates summary statistics on the number of WSIs, distinct patients, and annotations curated from TCGA and additional datasets, but the described systems and methods are not so limited. Next, cell- and tissue-type model predictions were exhaustively generated for 2,826 TCGA WSIs, which were then used to compute a diverse array of HIFs per WSI. It is appreciated that a plurality of WSI's may be used for a patient, and that an array of HIFs may be determined per patient.

### Cell- and Tissue-Type Predictions Yield a Wide Spectrum of HIFs

In some embodiments, in the first step of the pipeline, two Convolutional Neural Networks (CNNs) were trained per cancer type: (1) tissue-type models trained to segment cancer tissue, cancer-associated stroma, and necrotic tissue regions, and (2) cell-type models trained to detect lymphocytes, plasma cells, fibroblasts, macrophages, and cancer cells. These models were improved iteratively through a series of quality control steps, including significant input from board-certified pathologists (Methods). These CNNs were then used to exhaustively generate cell-type labels and tissue-type segmentations for each WSI. These predictions may be visualized as colored heatmaps projected onto the original WSIs. FIG. 5C illustrates unprocessed portions of STAD H&E-stained slides alongside corresponding heatmap visualizations of cell- and tissue-type predictions. Slide regions are classified into tissue types: cancer tissue (red), cancer-associated stroma (orange), necrosis (black), or normal (transparent). Pixels in cancer tissue or cancer-associated stroma areas are classified into cell types: lymphocyte (green), plasma cell (lime), fibroblast (orange), macrophage (aqua), cancer cell (red), or background (transparent). When quantified, these predictions may capture broad multivariate information about the spatial distribution of cells and tissues in each slide.

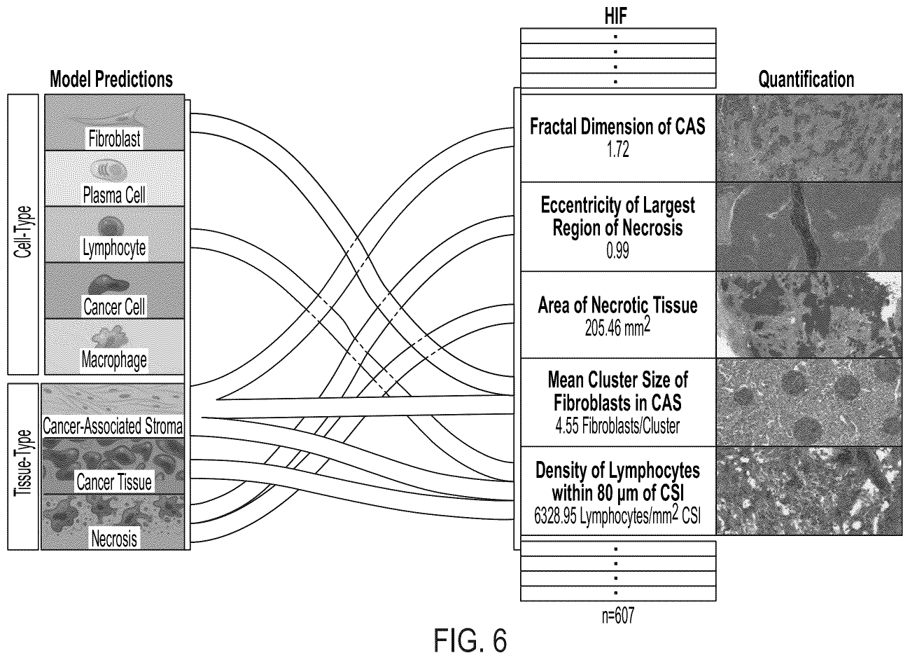

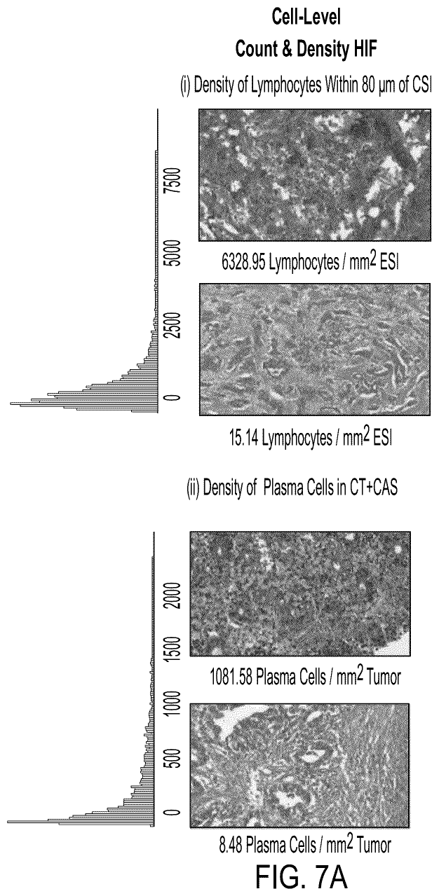

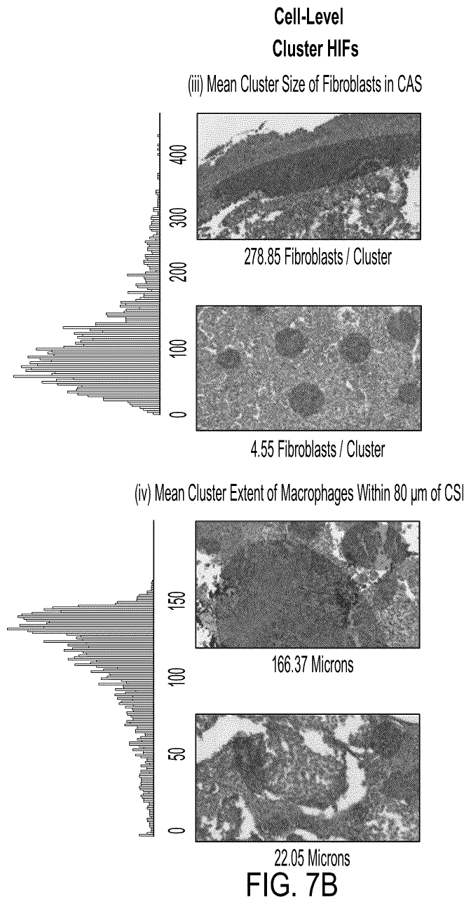

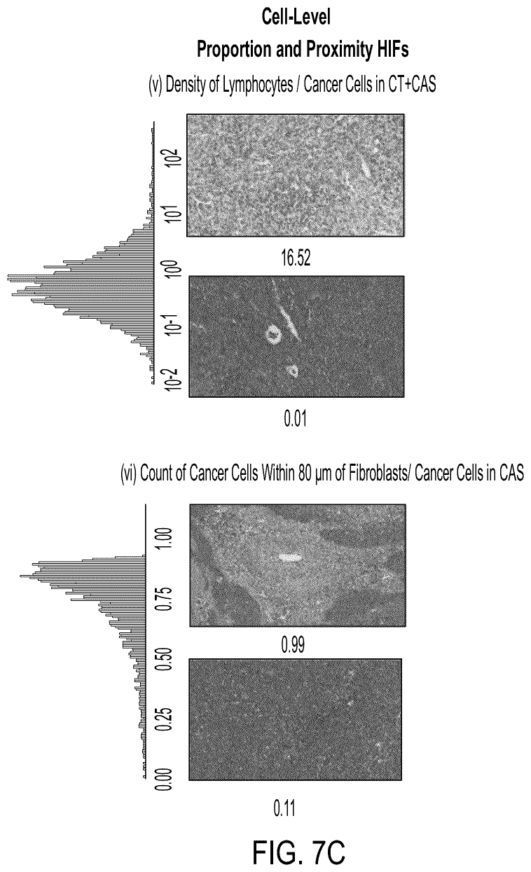

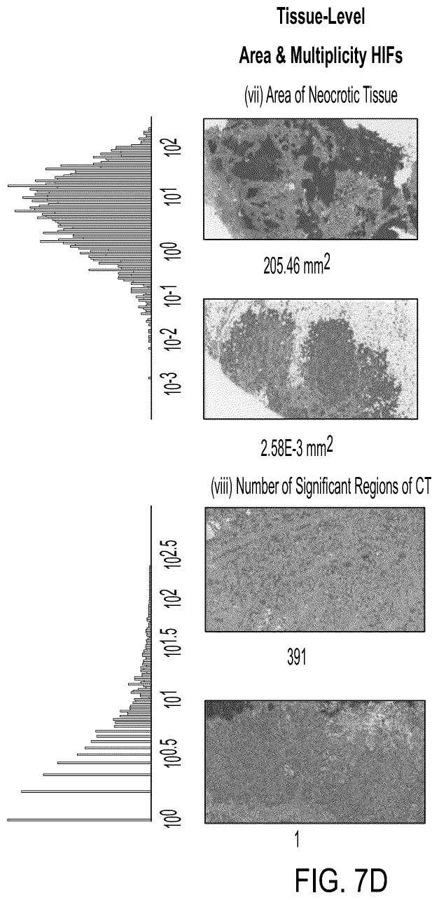

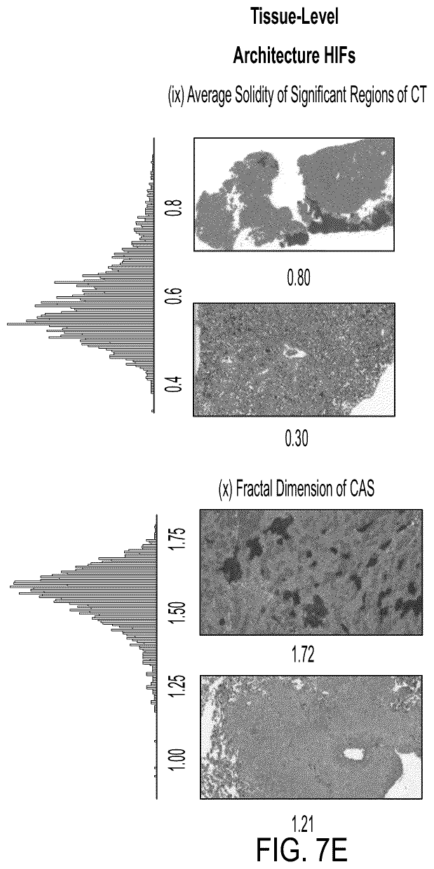

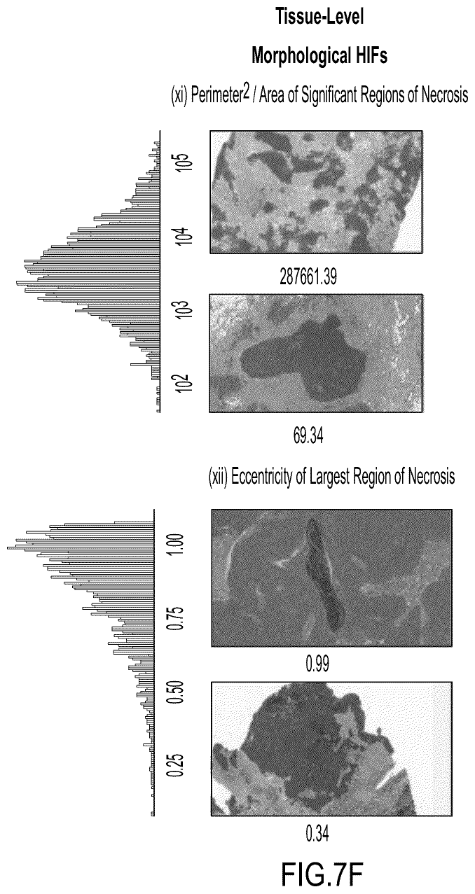

In an illustrative example, model predictions were used to extract 607 HIFs. FIG. 6 is a flow diagram of HIF extraction from model predictions for five example HIFs. For each HIF, an H&E snapshot with the corresponding cell- or tissue-type heatmap overlaid and the associated quantity are shown, which can be understood in terms of six categories. FIGS. 7A-7F illustrate a graphical overview of the 607 HIFs grouped into six categories: cell-level count and density (n=56 HIFs), cell-level cluster (n=180), cell-level proportion and proximity (n=208), tissue-level area and multiplicity (n=13), tissue-level architecture (n=25), and tissue-level morphological (n=125). For each HIF, a histogram of the HIF quantified in all patient samples across the five cancer types, and H&E snapshots corresponding to high and low values with the corresponding cell- or tissue-type heatmap overlaid are shown. Both snapshots are taken from patient samples of the same cancer type. Cell- and tissue-type heatmaps adhere to the same color scheme described in FIG. 5C. In (iii), fibroblast clusters are annotated, contrasting one large cluster against multiple smaller clusters. In (iv), macrophage clusters and extents are annotated. Cluster extent is defined as the maximum distance between a cluster exemplar (defined via Birch clustering) and a cell within that cluster. Significant regions (viii) are defined as connected components (identified at the pixel-level) of a given tissue type with at least 10% the size of the largest connected component in the slide. A solidity (ix) of one corresponds to a fully solid object, while values less than one correspond to objects containing holes or with irregular boundaries. Fractal dimension (x) can efficiently estimate the geometrical complexity and irregularity of shapes and patterns, thus capturing tissue architecture. A fractal dimension of one corresponds to a tissue border that is virtually smooth (a perfect line), while increasing fractal dimension corresponds to increasing roughness and irregularity, which translates into more extensive physical contact between adjacent tissue types. The fractal dimension of the CSI may be associated with dysfunction in antigen presentation. Perimeter²/Area (xi) is a unitless measure of shape roughness (e.g., square=16, circle=4π). Across all HIFs, tumor regions include cancer tissue (CT), cancer-associated stroma (CAS), and a combined CT+CAS.). The first category includes cell type counts and densities across different tissue regions (e.g. density of plasma cells in cancer tissue) (FIGS. 7A i-ii). The next category includes cell-level cluster features that capture inter-cellular spatial relationships, such as cluster dispersion, size, and extent (e.g., mean cluster size of fibroblasts in cancer-associated stroma) (FIGS. 7B iii-iv). The third category captures cell-level proportion and proximity features, such as the proportional count of lymphocytes versus fibroblasts within 80 microns (μm) of the cancer-stroma interface (CSI) (FIGS. 7C v-vi). The fourth category includes tissue area (e.g., mm² of necrotic tissue) and multiplicity counts (e.g. number of significant regions of cancer tissue) (FIGS. 7D vii-viii). The fifth category includes tissue architecture features, such as the average solidity (“solidness”) of cancer tissue regions or the fractal dimension (geometrical complexity) of cancer-associated stroma (FIGS. 7E ix-x). The final category captures tissue-level morphology using metrics such as perimeter² over area (shape roughness), lacunarity (“gappiness”), and eccentricity (FIGS. 7F xi-xii). This broad enumeration of biologically-relevant HIFs may enable unbiased exploration of mechanisms underlying histopathology across diverse cancer types.

### HIFs Capture Sufficient Information to Stratify Cancer Types

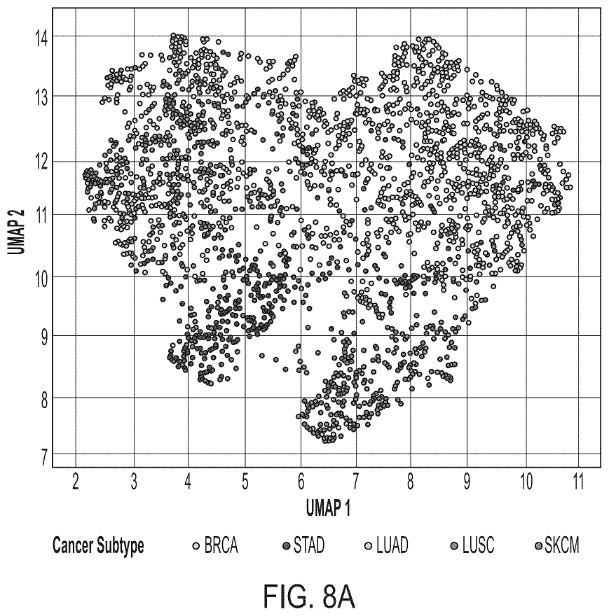

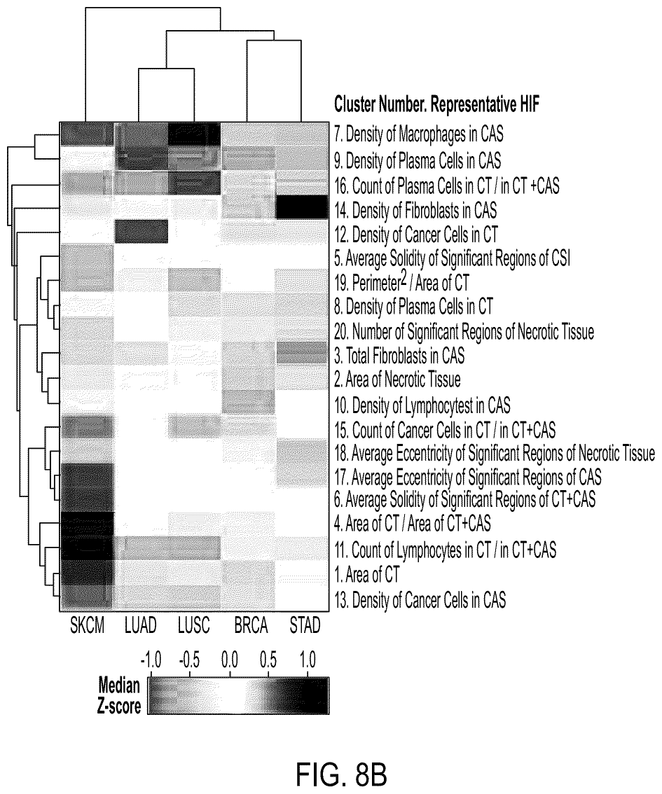

In some embodiments, to visualize the global structure of the HIF feature matrix Uniform Manifold Approximation and Projection (UMAP) was used to reduce the 607-dimensional HIF space into two dimensions. FIG. 8A illustrates UMAP projection and visualization of five cancer types reduced from the 607-dimension HIF space into two dimensions. Each point represents a patient sample colored by cancer type. The 2-D manifold projection of HIFs was able to separate BRCA, SKCM, and STAD into distinct clusters, while merging NSCLC subtypes LUAD and LUSC into one overlapping cluster (V-measure score=0.47 using k-means with k=4).

Cancer type differences could be traced to specific and interpretable cell- and tissue-level features within the TME. FIG. 8B illustrates a clustered heatmap of median Z-scores (computed pan-cancer) across cancer types for twenty HIFs, each representing one HIF cluster (defined pan-cancer). Hierarchical clustering was done using average linkage and euclidean distance. Clusters are annotated with a representative HIF chosen based on interpretability and high variance across cancer types. SKCM samples exhibited higher densities of cancer cells in cancer-associated stroma (pan-cancer median Z-score=0.55, P<10⁻³⁰) and greater cancer tissue area per slide (Z-score=0.72, P<10⁻³⁰) relative to other cancer types. These findings reflect biopsy protocols for SKCM, in which the excised region involves predominantly cancer tissue and minimal normal tissue. NSCLC subtypes LUAD and LUSC exhibited higher densities of macrophages in cancer-associated stroma (Z-score=0.54 and 0.91, respectively; P<10⁻³⁰), reflecting the large population of macrophages infiltrating alveolar and interstitial compartments during lung inflammation. NSCLC subtypes also exhibited higher densities of plasma cells (Z-score=0.61 and 0.49; P<10⁻³⁰) in cancer-associated stroma, in agreement with prior findings in which proliferating B cells were observed in ˜35% of lung cancers. STAD exhibited the highest density of lymphocytes in cancer-associated stroma (Z-score=0.11, P=2.16×10⁻¹⁹), corroborating prior findings which identified STAD as having the largest fraction of TIL-positive patches per WSI among thirteen TCGA cancer types, including the five examined thus far. Notably, HIFs are able to stratify cancer types by known histological differences without explicit tuning for cancer type detection.

HIFs are Concordant with Sequencing-Based Cell and Immune Marker Quantifications

In some embodiments, to further validate the deep learning-based cell quantifications, the abundance of the same cell type predicted by the cell-type models were compared with those based on RNA sequencing. Image-based cell counts were correlated with sequencing-based cell quantifications across all patient samples and cancer types (pan-cancer) in three cell types: leukocyte fraction (Spearman correlation coefficient=0.55, P<2.2×10⁻¹$), lymphocyte fraction (0.42, P<2.2×10⁻¹⁶), and plasma cell fraction (0.40, P<2.2×10⁻¹⁶). Notably, perfect correlation is not expected as tissue samples used for RNA sequencing and histology imaging are extracted from different portions of the patient's tumor, and thus vary in TME due to spatial heterogeneity.

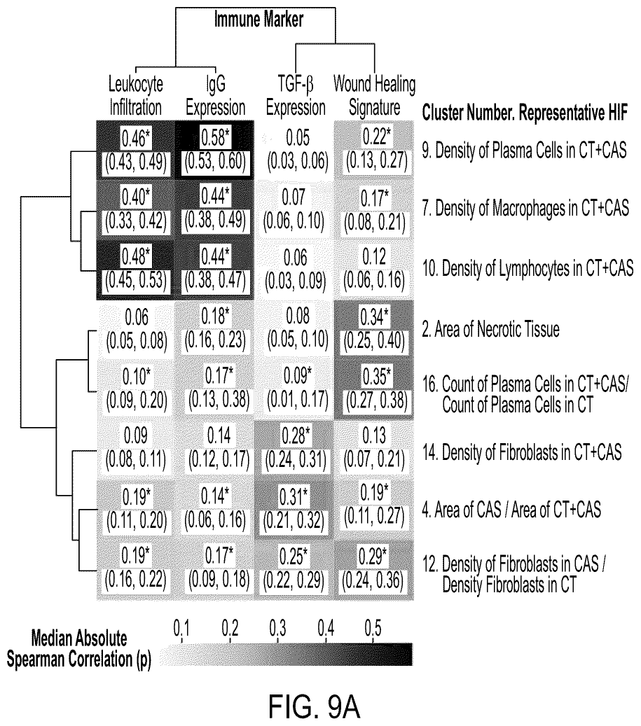

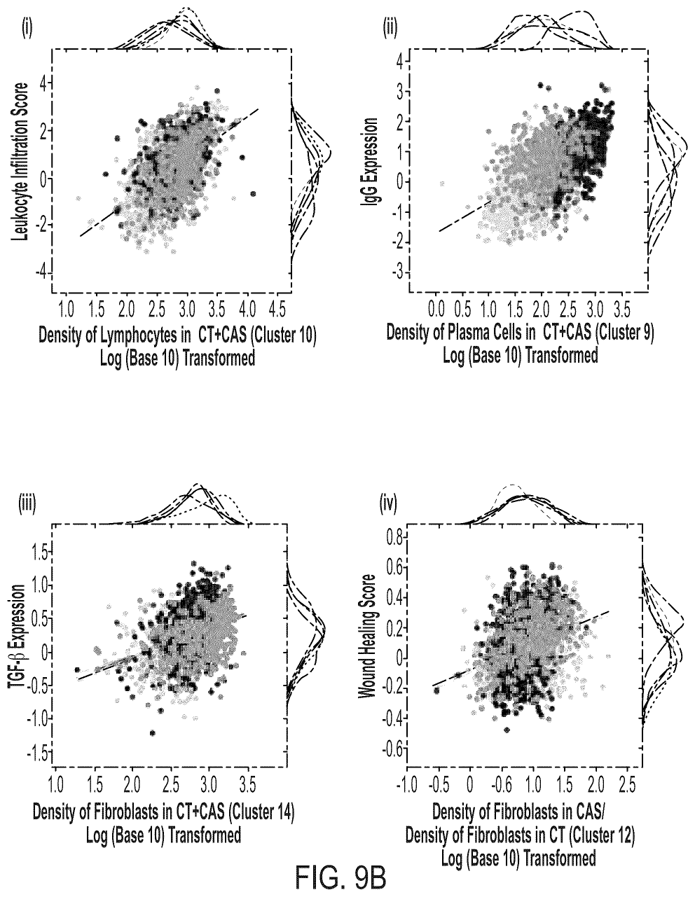

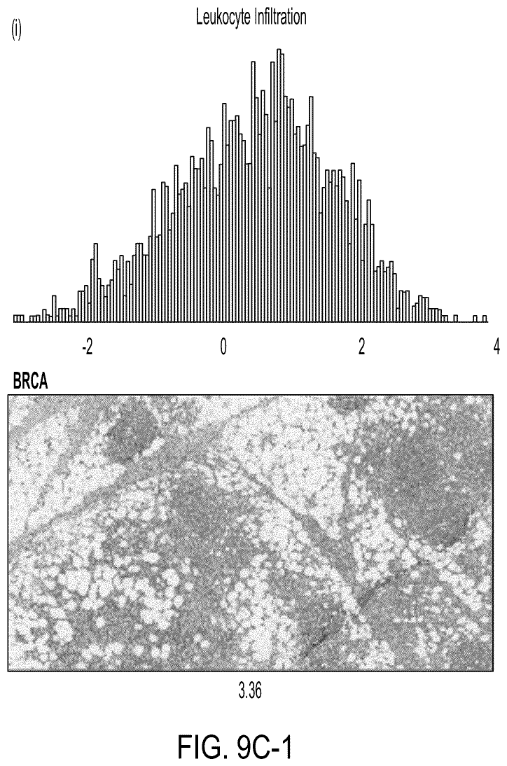

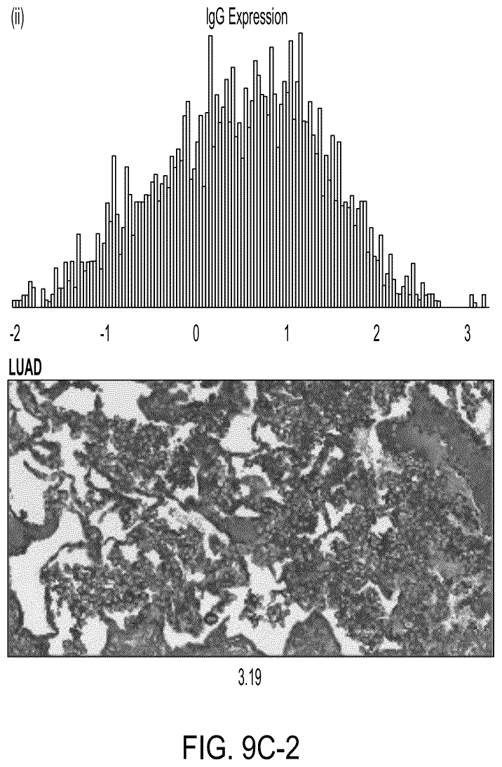

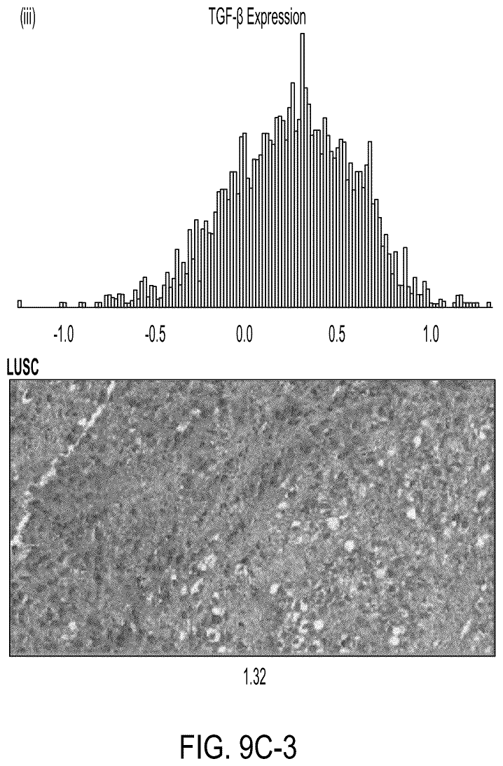

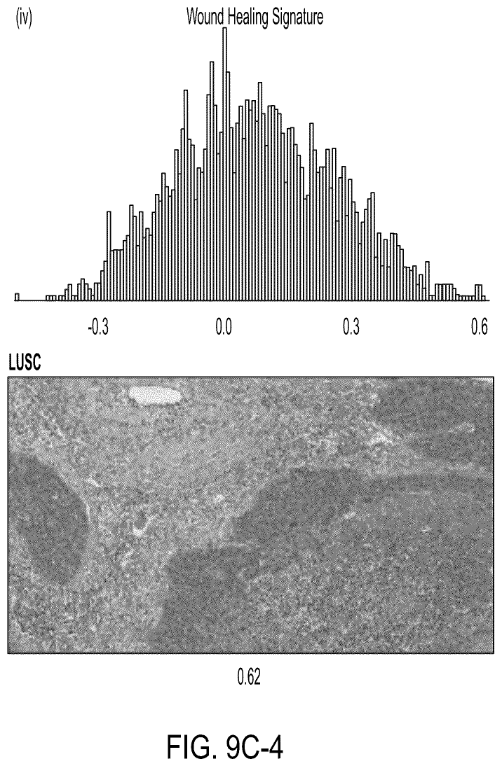

The inventors discovered there is significant correlation structure among individual HIFs due to the process by which feature sets are generated, as well as inherent correlations in the underlying biological phenomena. For example, proportion, density, and spatial features of a given cell or tissue type all rely on the same underlying model predictions. In order to identify mechanistically-relevant and inter-correlated groups of HIFs, hierarchical agglomerative clustering was conducted (Methods). This clustering also enables more accurate multiple-hypothesis-testing corrections, which account for feature correlation. Pan-cancer HIF clusters strongly correlated with immune markers of leukocyte infiltration, IgG expression, TGF-β expression, and wound healing. FIG. 9A illustrates a clustered heatmap of median absolute Spearman correlation coefficients (p) computed across all patient samples between eight HIF clusters (defined pan-cancer) and four canonical immune markers. Hierarchical clustering was done using average linkage and euclidean distance. Median absolute Spearman correlation coefficients with a combined (via the Empirical Brown's method) and corrected (via the Benjamini-Hochberg procedure) P value lower than the machine precision level (1×10⁻³⁰) are annotated with an asterisk. Tumor regions include cancer tissue (CT), cancer-associated stroma (CAS), and a combined CT+CAS, each quantified by scoring bulk RNA sequencing reads for known immune expression signatures. The same correlational analysis was conducted for each cancer type individually, and high concordance was observed among the top-correlated HIF clusters per immune marker.

Molecular quantification of leukocyte infiltration was concordant with the density of leukocyte-lineage cells in cancer tissue plus cancer-associated stroma (CT+CAS) quantified by the deep learning pipeline. FIG. 9B illustrates correlation and kernel density estimation plots between representative HIFs and immune markers. Points are colored by cancer type. X-axes are log-transformed (base ten). Trendlines are plotted on the log-transformed data. Cell densities are reported in count/mm² and tissue areas are reported in mm².), including lymphocytes (median absolute Spearman correlation p for associated HIF cluster=0.48, P<10⁻³⁰; FIG. 9B i), plasma cells (cluster ρ=0.46, P<10⁻³⁰), and macrophages (cluster ρ=0.40, P<10⁻³⁰). Similarly, associations were observed between IgG expression and the density of leukocyte-lineage cells in CT+CAS, with plasma cells being the most strongly correlated (cluster ρ=0.58, P<10⁻³⁰), given their role in producing immunoglobulins (FIG. 9B ii). TGF-β expression was associated with the density of fibroblasts in CT+CAS (cluster ρ=0.28, P<10⁻³⁰; FIG. 9B iii), building upon prior studies which found that TGF-β1 can promote fibroblast proliferation. TGF-β expression was also correlated with the area of cancer-associated stroma relative to CT+CAS (cluster ρ=0.31, P<10⁻³⁰), shedding further light on the role of stromal proteins in modulating TGF-β levels. Wound healing signature was positively associated with the density of fibroblasts in cancer-associated stroma versus in cancer tissue (cluster ρ=0.29, P<10⁻³⁰; FIG. 9B iv), which corroborates findings that both tumors and healing wounds alike modulate fibroblast recruitment and proliferation to facilitate extracellular matrix deposition. H&E snapshots corresponding to high expression of each of the four immune markers are shown in FIGS. 9C-1-5C-4 with corresponding cell-type heatmaps overlaid. FIGS. 9C-1-9C-4 are histograms of immune marker expression (Z-score) across all patients, alongside an H&E snapshot with its cell-type heatmap overlaid corresponding to high expression of the given immune marker. Cell-type heatmaps adhere to the same color scheme described in FIG. 5C.

### HIFs are Predictive of Clinically-Relevant Phenotypes

In some embodiments, to evaluate the capability of HIFs to predict expression of clinically-relevant, immuno-modulatory genes, supervised prediction of binarized classes for five clinically-relevant phenotypes was conducted: (1) programmed cell death protein 1 (PD-1) expression, (2) PD-L1 expression, (3) cytotoxic T-lymphocyte-associated protein 4 (CTLA-4) expression, (4) HRD score, and (5) T cell immunoreceptor with Ig and ITIM domains (TIGIT) expression (FIGS. 10A-1-10B-2), but the described systems and methods are not so limited. In an illustrative example, using the 607 HIFs computed per WSI, predictions were conducted for cancer types individually as well as pan-cancer. SKCM predictions were conducted only for TIGIT expression due to insufficient sample sizes for the remainder of outcomes (Methods). To demonstrate model generalizability across varying patient demographics and sample collection processes, area under the receiver operating characteristic (AUROC) and area under the precision-recall curve (AUPRC) performance metrics were computed on hold-out sets composed exclusively of patient samples derived from tissue source sites not seen in the training sets.

HIF-based models may not be predictive for every phenotype in each cancer type. In the successful prediction models (e.g., hold-out AUROC range=0.601-0.864; FIGS. 10A-1-10B-2), precision-recall curves revealed that models were robust to class-imbalance, achieving AUPRC performance surpassing positive class prevalence by 0.104-0.306 (FIGS. 10A-1-10A-2; ROC curves for (i) PD-1, (ii) PD-L1, (iii) CTLA-4, (iv) HRD, and (v) TIGIT hold-out predictions across cancer types and pan-cancer. SKCM predictions were conducted only for TIGIT due to low sample sizes. Pan-cancer predictions use binary labels thresholded independently by cancer type. For TIGIT predictions, pan-cancer includes all five cancer types. For the remainder of predictions, pan-cancer includes all cancer types excluding SKCM. Random classifiers correspond to AUROC=0.50.). Notably, AUROC performance of the HIF-based linear model for PD-L1 expression in LUAD was comparable to that achieved by “black-box” deep learning models trained on hundreds of thousands of paired H&E and PD-L1 example patches in NSCLC.

### Predictive HIFs Provide Interpretable Link to Clinically-Relevant Phenotypes

In some embodiments, interpretable features may enable interrogation and further validation of model parameters as well as generation of new biological hypotheses. Towards this end, in some embodiments, for each prediction task the five most important HIF clusters were identified, as determined by magnitude of model coefficients. FIGS. 10B-1-10B-2 illustrate visualization of predictive HIFs for each molecular phenotype. Boxplots show the top five most predictive HIF clusters for each phenotype in pan-cancer models. For TIGIT predictions, pan-cancer models only included three non-zero HIF clusters. Clusters are ranked by the maximum absolute ensemble beta across HIFs in a given cluster. Ensemble betas are computed per HIF as the average across the three models incorporated into the final ensemble evaluated on the hold-out set. Each boxplot highlights the median and interquartile range for ensemble betas in each cluster. Each cluster is labeled with a representative HIF corresponding to the maximum absolute ensemble beta value. In cases where that HIF is difficult to interpret, a more interpretable HIF within a five-fold difference of the maximum ensemble beta is presented (indicated by a black asterisk). As absolute values were used for ranking, HIFs with negative ensemble betas are denoted by a red asterisk. Radar charts show the normalized magnitude of ensemble betas in pan-cancer models stratified across nine HIF axes, corresponding to the five cell types, three tissue types, and CSI. Normalized magnitudes were computed as the sum of absolute ensemble betas for HIFs associated with each axis divided by the total number of HIFs associated with said axis (e.g. all HIFs involving fibroblasts). Multiple predictive HIFs are visualized with overlaid cell- or tissue-type heatmaps in FIGS. 7A-7F. Tumor regions include cancer tissue (CT), cancer-associated stroma (CAS), and a combined CT+CAS.) and computed cluster-level P-values to evaluate significance (Methods).

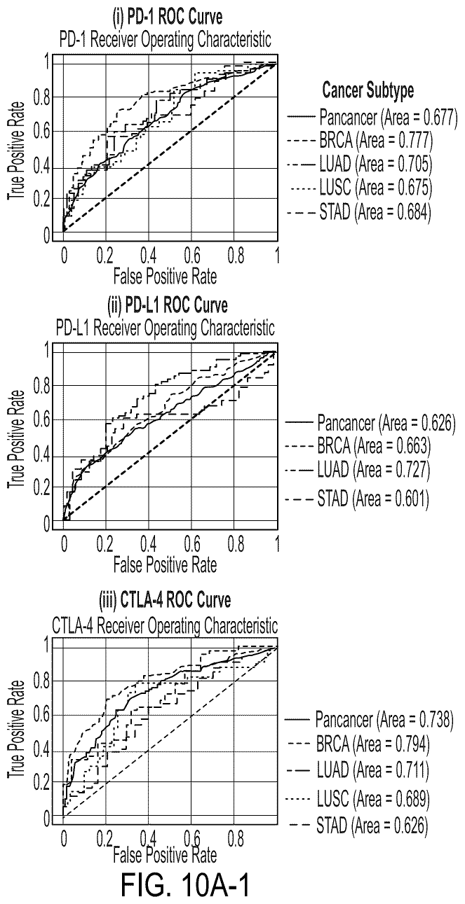

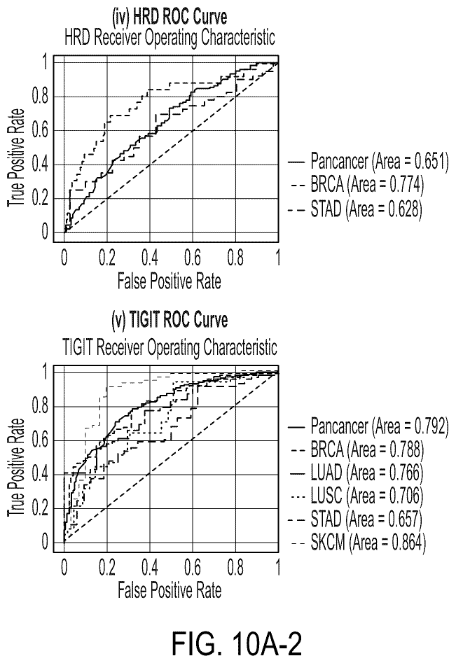

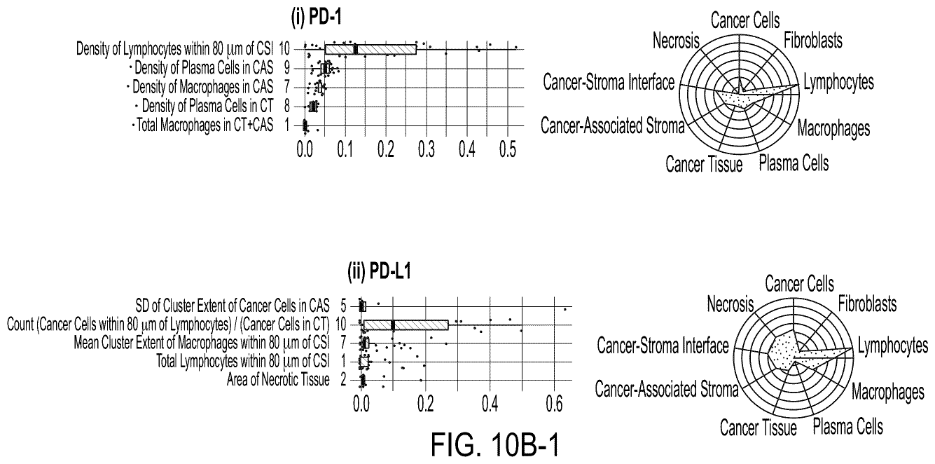

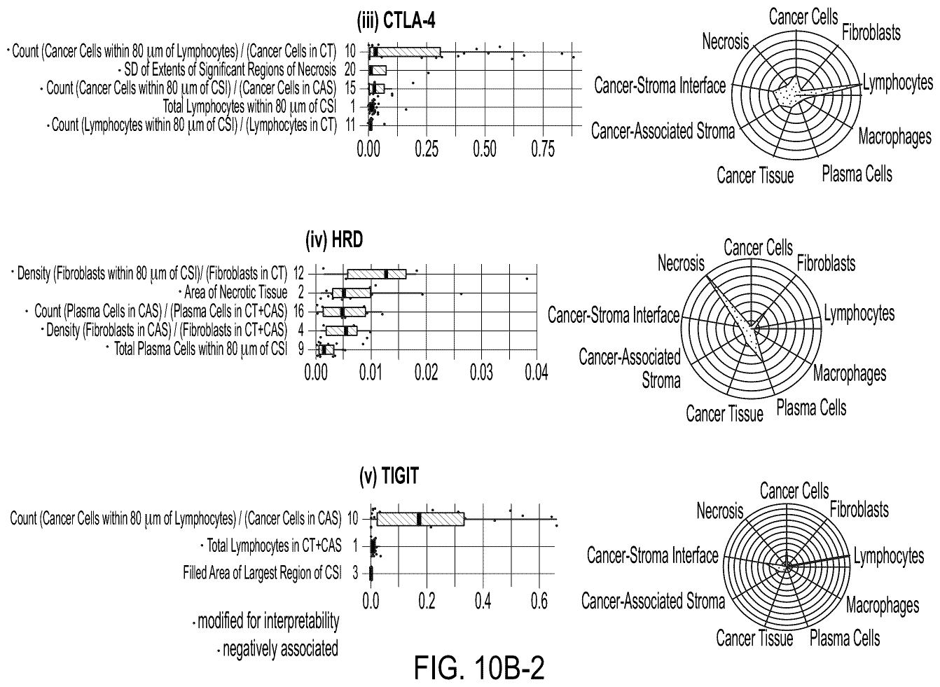

The inventors appreciated that prediction of PD-1 and PD-L1 involved similar HIF clusters (Pearson correlation between PD-1 and PD-L1 expression=0.53). For example, the extent of tumor inflammation, as measured by the count of cancer cells within 80 μm of lymphocytes, as well as the density of lymphocytes in CT+CAS, was significantly selected during model fitting for both of PD-1 and PD-L1 expression in pan-cancer and BRCA models (FIGS. 10B-1 i-ii). Furthermore, in both LUAD and LUSC, the count of lymphocytes in CT+CAS was similarly predictive of PD-1 and PD-L1 expression. The importance of these HIFs which capture lymphocyte infiltration between and surrounding cancer cells corroborates prior literature which demonstrated that TILs correlated strongly with higher expression levels of PD-1 and PD-L1 in early breast cancer and NSCLC.

The area, morphology, or multiplicity of necrotic tissue proved predictive of PD-1 expression in LUAD, LUSC, and STAD models and of PD-L1 expression in pan-cancer, BRCA, and LUAD models, expanding upon prior findings that tumor necrosis correlated positively with PD-1 and PD-L1 expression in LUAD. The density, proximity, or clustering properties of plasma cells was predictive of PD-1 expression in all models excluding LUAD, suggesting a role for plasma cells in modulating PD-1 expression. Recent studies in SKCM, renal cell carcinoma, and soft-tissue sarcoma have demonstrated that an enrichment of B-cells in tertiary lymphoid structures was positively predictive of response to immune checkpoint blockade therapy. The density of fibroblasts in cancer-associated stroma or within 80 μm of the CSI was predictive of PD-L1 expression in LUAD and STAD, respectively, corroborating earlier discoveries that cancer-associated fibroblasts promote PD-L1 expression.

Less is known about the relationship between the TME and CTLA-4 expression. By investigating predictive HIFs, features of the TME that correlate with CTLA-4 expression can be enumerated. The proximity of lymphocytes to cancer cells (pan-cancer and BRCA), morphology of necrotic regions (LUAD and LUSC), and density of cancer cells in CT+CAS versus exclusively in cancer-associated stroma (BRCA and STAD) were predictive of CTLA-4 expression across multiple models (FIG. 10B-2 iii).

Area of necrotic tissue (pan-cancer and BRCA) as well as various morphological properties of necrotic regions including perimeter and lacunarity (BRCA and STAD) were predictive of HRD (FIG. 10B-2 iv). In HRD, ineffective DNA damage repair can result in the accumulation of severe DNA damage and subsequent cell death through apoptosis as well as necrosis. The density and count of fibroblasts near or in cancer-associated stroma was also predictive of HRD in the pan-cancer and BRCA models, corroborating prior findings that persistent DNA damage and subsequent accumulation of unrepaired DNA strand breaks can induce reprogramming of normal fibroblasts into cancer-associated fibroblasts.

Like the three other immune checkpoint proteins (PD-1, PD-L1, and CTLA-4), TIGIT expression was also associated with markers of tumor inflammation, including the count of cancer cells within 80 μm of lymphocytes (pan-cancer and BRCA), the total number of lymphocytes in CT+CAS (pan-cancer and BRCA), and the proportional count of lymphocytes to cancer cells within 80 μm of the CSI (LUAD) (FIG. 10B-2 v). These findings corroborate prior findings that TIGIT expression, alongside PD-1 and PD-L1 expression (Pearson correlation between TIGIT and PD-1=0.84; TIGIT and PD-L1=0.56), is correlated with TILs. HIF clusters capturing morphology and architecture of necrotic tissue (e.g. fractal dimension, lacunarity, extent, perimeter²/area) were associated with TIGIT expression in LUAD, LUSC, SKCM, and STAD models, although these relationships have yet to be investigated.

### Discussion

The inventors' study is the first to demonstrate the value of combining deep learning-based cell- and tissue-type classifications to compute image features that are both biologically-relevant and human interpretable. The inventors demonstrate that computed HIFs can recapitulate sequencing-based cell quantifications, capture canonical immune markers such as leukocyte infiltration and TGF-β expression, and robustly predict five molecular phenotypes relevant to oncology treatment efficacy and response, but the described systems and methods are not so limited. It is appreciated that the human interpretable features framework can also be applied to other cancer types and/or therapy treatment, such as immunotherapy treatment for solid tumors. The inventors also demonstrate the generalizability of the associations, as evidenced by similarly predictive HIF clusters across biopsy images derived from five different cancer types. While prior studies have applied deep learning methodologies to capture cell-level information, such as the spatial configuration of immune and stromal cells, or tissue-level information alone, the combined cell plus tissue approach enables quantification of increasingly complex and expressive features of the TME, ranging from the mean cluster size of fibroblasts in cancer-associated stroma to the proximity of TILs or cancer-associated fibroblasts to the CSI. By training models to make six-class cell-type and four-class tissue-type classifications, the inventors' approach is also able to aggregate more layers of information than prior studies, but the described systems and methods are not so limited. Indeed, while TILs are emerging as a promising biomarker in solid tumors such as triple-negative and HER2-positive breast cancer, TILs differ from stromal lymphocytes, and substantial signal can be obtained by considering multiple cell-tissue combinations.

The inventors' approach of exhaustively generating cell- and tissue-type predictions across entire WSIs at a spatial resolution of two and four μm, respectively is novel and improves upon previous tiling approaches that downsample the image and subsequently remove valuable information. However, the described systems and methods are not so limited and may be equally applicable at other spatial resolutions. The tissue visible in a WSI is already only a fraction of the tumor itself, and using the entire slide (rather than selected tiles) reduces the probability of fixating on non-generalizable local effects and enables quantification of complex characteristics that span multiple tissue regions (e.g. multiplicity, solidity, and fractal dimension of significant necrotic regions).

In addition, the inventors' approach of capturing specific and interpretable features of the tumor and its surroundings can facilitate hypothesis generation and enable a deeper understanding of the TME's influence on drug response. Indeed, recent studies provide evidence that tumor immune architecture can greatly dictate clinical efficacy of immune checkpoint inhibitor and poly (ADP-ribose) polymerase (PARP) inhibitor therapies.

Lastly, during both model development and evaluation, the inventors sought to emphasize robustness to real-world variability. In particular, TCGA WSIs were supplemented with additional diverse datasets during CNN training, pathologist feedback was integrated into model iterations, and HIF-based model performance was evaluated on hold-out sets composed exclusively of samples from unseen tissue source sites, improving upon prior approaches to predicting molecular outcomes from TCGA H&E images,

One limitation of machine-learning approaches can be the quality of training data. While the cell and tissue classification models can be trained on a combination of TCGA and additional datasets, molecular associations and predictions may be derived solely from TCGA. Biopsy images submitted to the TCGA dataset suffer from selection bias towards more definitive diagnoses and early-stage disease that may not generalize well to ordinary clinical settings. Moreover, the images only contain H&E staining, which can limit the amount of information available. It is possible that integrating multimodal data containing stains against Ki-67 or immunohistological targets may increase confidence in cell classifications. In addition to the quality of slide images, annotations are also variable in reliability. Macrophages are particularly difficult for pathologists to identify solely under H&E staining. While the accuracy of an individual pathologist identifying macrophages may be poor, the models described herein represent a consensus across hundreds of pathologist annotators which may carry a more reliable signal.

Furthermore, morphologically similar cells (e.g. macrophages, dendritic cells, endothelial cells, pericytes, myeloid derived suppressor cells, and atypical lymphocytes) may all be captured under a single cell-type prediction. Thus, HIFs may, in reality, capture information about a mixture of cell types. For example, in diffuse forms of STAD in which cancer cells invade smooth muscle tissue, the models may misclassify certain smooth muscle cells as fibroblasts. Therefore, fibroblast-label HIFs may reflect a mixture of these two cell types in STAD, limiting interpretability. Iterative model training coupled with pathologist evaluation could have mitigated but likely not eliminated this cell type confusion. Nonetheless, these features were recurrently selected for prediction of PD-L1 and CTLA-4 expression in STAD, possibly demonstrating robustness to misclassification noise. The accuracy of the model predictions can be sufficiently robust depending on that computed HIFs correlate with the true underlying features.

These interpretable sets of HIFs as described in various embodiments, as computed from, e.g., tens-of-thousands of deep learning-based cell- and tissue-type predictions per patient, may be central to the value of HIF-based models. Such models improve upon conventional “black-box” approaches which apply deep learning directly to WSIs, yielding models with millions of parameters and limited interpretability. Recent findings have revealed the weaknesses of low-interpretability models, including brittleness to dataset shift, vulnerabilities to adversarial attack, and susceptibility to the biases of the data-generative process. Beyond suggesting interpretable hypotheses for causal mechanisms (e.g., the anti-tumor effect of high lymphocyte density), the HIF-based approach can be continually validated at several points: pathologists can judge the quality of cell and tissue-type predictions, estimate the values of each relevant feature using traditional manual scoring, and observe whether there is a significant failure given real-world variability in sample preparation and quality. While “black-box” models may opaquely rely on features that are predictive but disconnected from the outcome of interest, such as tissue excision or preparation artifacts (e.g. surgical or pathologist markings), relationships underlying HIF-based predictions can be traced to specific variables, allowing model failures to be explained and addressed. While empirical performance is vitally important in clinical settings and additional studies comparing end-to-end and HIF-based approaches are needed, the improved trust and reliability against unexpected failures make HIF-based models a valuable, and potentially preferable, alternative.

Finally, the ability to predict molecular phenotypes directly from WSIs in an interpretable fashion has numerous potential benefits for clinical oncology. Hospitals, healthcare institutions, and pharmaceutical and biotechnology companies have decades of archival histopathology data captured from routine care and clinical trials. HIF-based models capable of capturing molecular information can supplement molecular assays that are often expensive and time-consuming, enable the discovery of novel patient sub-populations with specific disease processes and treatment susceptibilities, and generate hypotheses that can guide subsequent pre-clinical and clinical research.

### Methods: Dense, High-Resolution Prediction of Cell and Tissue Types Using Convolutional Neural Networks

In some embodiments, in order to compute histopathological image features for each slide, first cell and tissue predictions per WSI are generated. To this end, a network of board-certified pathologists was asked to label WSIs with both polygonal region annotations based on tissue type (cancer tissue, cancer-associated stroma, necrotic tissue, and normal tissue or background) and point annotations based on cell type (cancer cells, lymphocytes, macrophages, plasma cells, fibroblasts, and other cells or background). This collection of expert annotations was then used to train six-class cell type and four-class tissue-type classifiers.

Several steps were taken to ensure the accuracy and generalizability of the models described herein. First, it was important to recognize that common cell and tissue types, such as cancer-associated stroma or cancer cells, show morphological differences between BRCA, LUAD, LUSC, SKCM, and STAD. As a result, separate cell- and tissue-type detection models were trained for each of these five cancer types, for a total of ten models. Second, it was important to ensure that the models did not overfit to the histological patterns found in the training set. To avoid this, the data was split into training, validation, and test sets, and incorporated annotated slides of the same five cancer types into the model development process. Together, these datasets represented a wide diversity of examples for each class in each cancer type, thus improving the generalizability of these models beyond the TCGA dataset.

Using the combined dataset of annotated TCGA and additional WSIs, deep Convolutional Neural Networks (CNN) were trained to output dense pixelwise cell- and tissue-type predictions at a spatial resolution of two and four μm, respectively (e.g., spatial resolution dictated by stride). To ensure that the models achieved sufficient accuracy for feature extraction, models were trained in an iterative process, with each updated model's predictions visualized as heatmaps to be reviewed by board-certified pathologists. In heatmap visualizations, tissue regions were segmented and filled by different colors, while cell types were represented by different-colored squares. This process continued until there were minimal systematic errors and the pathologists deemed the model sufficiently trustworthy for feature extraction.

### Pathologist Validation of Cell- and Tissue-Type Predictions

In some embodiments, during the CNN training process, three board-certified pathologists were asked to iteratively conduct subjective evaluation of model predictions to inform multiple rounds of training. CNN models were initially trained on a set of primary annotations collected from the pathologist network. Following the conclusion of each training round (defined by model convergence), predicted cell and tissue heatmaps were reviewed for systematic errors (e.g. overprediction of fibroblasts, macrophages, and plasma cells, underprediction of necrotic tissue). New annotations would then be collected from the pathologist network focusing on areas of improvement (e.g. mislabeled macrophages) to initiate a subsequent training round.

### Tissue-Based Feature Extraction

In some embodiments, using the tissue-type predictions, 163 different region-based features were extracted from each WSI in the TCGA dataset. Each of these features belonged to one of three general categories.

The first category consisted of areas (n=13 HIFs). By simple pixel summation, the total areas (in mm²) of cancer tissue, cancer-associated stroma, cancer tissue plus cancer-associated stroma, regions at the cancer-stroma interface, and necrosis in each slide were computed. These numbers represent prime examples of features that are interpretable and technically attainable by human pathologists, but would be prohibitively time-consuming and inconsistent across pathologists to calculate in practice.

The second category, which contributed the bulk of the features, made use of the publicly available scikit-image.measure.regionprops module to find the connected components of each of these tissue types at the pixel-level using eight-connectivity. Once these connected components were found, a series of both library-provided and self-implemented methods were used to extract a series of morphological features (n=125 HIFs). These HIFs measured a wide variety of tissue characteristics, ranging from quantitative, size-based measures like the number of connected components, major and minor axis lengths, convex areas, and filled areas, to more qualitative, shape-based measures like Euler numbers, lacunarity, and eccentricity. Recognizing the log-distribution of connected component size, these features were computed not just across all connected components, but also for both the largest connected component only and across the most “significant” connected components, defined as components larger than 10% the size of the largest connected component. In aggregating metrics across considered components, both averages and standard deviations of HIFs were incorporated (e.g. standard deviation of eccentricities of significant regions of necrosis), to capture both summary metrics and metrics of intratumor heterogeneity.

The third category of features captures tissue architecture (n=25 HIFs). The fractal dimensions and solidity measures of different tissue types were calculated, capturing both the roundness and filled-ness of the tissue, under the hypothesis that the ability for these measures to separate different subtypes of lung cancer might translate to a similar ability to predict clinically-relevant phenotypes. In addition, these features allowed for capture of information about how tissue filled up space, rather than just the summative sizes and shapes captured by the first and second categories.

### Cell- and Tissue-Based Feature Extraction

In some embodiments, after obtaining six-class cell-type predictions for each pixel of a WSI, generated five binary masks corresponding to each of the five specified cell types were generated. The cell- and tissue-level masks were combined to compute properties of each cell type in each tissue type (e.g. fibroblasts in cancer-associated stroma), extracting 444 HIFs.

An initial group of features that were readily calculable from the model predictions included simple counts and densities of cell types in different tissue types. For example, an overlay of a particular slide's lymphocyte detection mask on top of the same slide's cancer-associated stroma mask could be used to calculate the number of TILs on a given slide. This number could then be divided by the area of cancer-associated stroma to find the associated density of TILs on the slide. By “taking the outer product” of cell and tissue types, a wide array of composite features can be derived. In particular, counts, proportions, and densities of cells across different tissue types were calculated (e.g. density of macrophages in cancer-associated stroma versus in cancer tissue), under the hypothesis that these measures capture information that raw counts could not. To capture information regarding cell-cell proximity and interactions, counts and proportions of each cell type were also calculated within an 80 μm radius of each other cell type (e.g. count of lymphocytes within an 80 μm radius of fibroblasts). Cell-level counts, densities, and proportions comprised 264 HIFs.

For each cell-tissue combination, the Birch clustering method was applied (as implemented in the sklearn.cluster Python module) to partition cells into clusters. To fit clustering structures as closely as possible to the spatial relationships found between cell types on the slide, a threshold of 100 was set, a branching factor of 10 was set, and the algorithm was allowed to optimize the number of clusters returned. The returned clusters were used to calculate a series of features designed to capture spatial relationships between individual cells types within a given tissue type, including number of clusters, cluster size mean and standard deviation (SD), within-cluster dispersion mean and SD, cluster extent mean and SD, the Ball-Hall Index, and Calinski-Harabasz Index (n=180 HIFs). For metrics where cluster exemplars were needed, the subcluster centers returned by the Birch algorithm were used.

### Patient-Level Aggregation

In some embodiments, patients with multiple tissue samples were represented by the single sample with the largest area of cancer tissue plus cancer-associated stroma, computed during tissue-based feature extraction. All subsequent analyses were conducted at the patient level.

### HIF Clustering

In some embodiments, due to underlying biological relationships as well as the HIF generation process, there is significant correlation structure between many of the features. This may present a challenge of feature selection as much of the information contained in one feature will also be present in another. It may also make it difficult to control for multiple hypothesis testing, because the underlying number of tested hypotheses is significantly fewer than the number of features computed.

To identify groups of correlated HIFs, features were clustered via hierarchical agglomerative clustering using complete linkage, a cluster cutoff of 0.95, and pairwise (1−absolute Spearman correlation) as the distance metric. A set of HIF clusters was defined for each cancer type independently, as well as another set for pan-cancer analyses. Clustering correlated features allows for summarizing the true underlying number of tested hypotheses.

### Visualization of Cancer Types in HIF Space

In some embodiments, Uniform Manifold Approximation and Projection (UMAP) was applied for dimensionality reduction and visualization of patient samples from the 607-dimension HIF space into two dimensions (using parameters: number of neighbors=15, training epochs=500, distance metric=euclidean). The V-Measure was computed to compare BRCA, STAD, SKCM, and NSCLC (LUAD and LUSC combined) classes against clusters generated by K-means (k=4) applied to the 2-D UMAP projection. To quantify differences between cancer types, HIF values were normalized pan-cancer into Z-scores. Median Z-scores were then computed per cancer type across twenty HIFs, each representing one of twenty HIF clusters defined pan-cancer. Representative HIFs were selected based on subjective interpretability and high variance across cancer types. To determine the statistical significance of median Z-scores that were greater in one cancer type relative to others, P-values were estimated with the one-sided Mann-Whitney U-test, considering NSCLC subtypes LUAD and LUSC as one type.

### Validation of HIFs Against Molecular Markers

To validate the ability of HIFs to capture meaningful cell- and tissue-level information, Spearman correlations between HIFs and four canonical immune markers from the PanImmune dataset were computed: (1) leukocyte infiltration, (2) IgG expression, (3) TGF-β expression, and (4) wound healing. Immune markers were quantified by mapping mRNA sequencing reads against gene sets associated with known immune expression signatures. To estimate the correlation between HIF clusters and immune markers, the median absolute Spearman correlation per cluster and combined dependent P-values associated with individual correlations via the Empirical Brown's method were computed. To control the false discovery rate, combined P-values per cluster were then corrected using the Benjamini-Hochberg procedure. Correlation analyses were conducted for cancer types collectively and individually, using HIF clusters defined across all cancer types for assessment of concordance.

In addition, image-based cell quantifications for leukocyte fraction, lymphocyte fraction, and plasma cell fraction were validated by Spearman correlation to their sequencing-based equivalents from matched TCGA tumor samples, computed using CIBERSORT. CIBERSORT (cell-type identification by estimating relative subsets of RNA transcripts) uses an immune signature matrix to deconvolve observed RNA-Seq read counts into estimates of relative contributions between 22 immune cell profiles.

### Molecular Phenotype Label Curation

In some embodiments, PD-1, PD-L1, and CTLA-4 expression data for each cancer type were collected from the PanImmune dataset, while TIGIT expression data was collected from the National Cancer Institute Genomic Data Commons. PD-1, PD-L1, CTLA-4, and TIGIT expression levels were quantified from mapped mRNA reads against genes PDCD1, CD274, CTLA-4, and TIGIT, respectively, and normalized as Z-scores across all cancer types in TCGA. Homologous recombination deficiency (HRD) scores were collected from Knijnenburg et al. The HRD score was calculated as the sum of three components: 1) number of subchromosomal regions with allelic imbalance extending to the telomere, 2) number of chromosomal breaks between adjacent regions of least 10 Mb, and 3) number of loss of heterozygosity regions of intermediate size (>15 Mb, but <whole chromosome length). Continuous immune checkpoint protein expression and HRD scores were binarized to high versus low classes using gaussian mixture model (GMM) clustering with unequal variance. The binary threshold was defined as the intersection of the empirical densities between the two GMM-defined clusters. To evaluate the extent to which prediction tasks were correlated, Pearson correlation and percentage agreement metrics were computed pan-cancer (n=1,893 patients) between the five molecular phenotypes in continuous and binarized form, respectively.

### Hold-Out Set Definition by TCGA Tissue Source Site

In some embodiments, TCGA may provide tissue source site information, which denotes the medical institution or company that provided the patient sample. For each prediction task (described below), a hold-out set was defined as approximately 20-30% of patient samples obtained from sites not seen in the training set. This validation methodology enables us to demonstrate model generalizability across varying patient demographics and tissue collection processes intrinsic to different tissue source sites.

### Supervised Prediction of Molecule Phenotypes

In some embodiments, supervised prediction of binarized high versus low expression of five clinically-relevant phenotypes was conducted: (1) PD-1 expression, (2) PD-L1 expression, (3) CTLA-4 expression, (4) HRD score, and (5) TIGIT expression. Predictions were conducted pan-cancer as well as for cancer types individually. SKCM was excluded from prediction tasks 1-4 due to insufficient outcome labels (number of observations<100 for tasks 1-3; number of positive labels<10 for task 4). For each prediction task, a logistic sparse group lasso (SGL) model was trained and tuned by nested cross validation (CV) with three outer folds and five inner folds using the corresponding training set. SGL provides regularization at both an individual covariate (as in traditional lasso) and user-defined group level, thus encouraging group-wise and within group sparsity. The HIF clusters defined per cancer type and pan-cancer (previously described) were inputted as groups. HIFs were normalized to mean=0 and SD=1. In accordance with nested CV, hyper-parameter tuning was conducted using the inner loops and mean generalization error and variance were estimated from the outer loops. The three tuned models, each trained on two of the three outer folds and evaluated on the third outer fold, were ensembled by averaging predicted probabilities for final evaluation (reported in FIGS. 6A-1-6A-2) on the hold-out set. Hold-out performance was evaluated by AUROC and AUPRC. To identify predictive features, beta values from the three outer fold models were averaged to obtain ensemble beta values per HIF (see FIGS. 6B-1-6B-2 caption for more details).

### Statistical Analysis

In some embodiments, to compute 95% confidence intervals for each prediction task, empirical distributions of AUROC and AUPRC metrics were generated, each consisting of 1000 bootstrapped metrics. Bootstrapped metrics were obtained by sampling with replacement from matched model predictions (probabilities) and true labels for the corresponding hold-out set, and re-computing AUROC and AUPRC on these two bootstrapped vectors. P-values for ensemble beta values of predictive HIFs were computed using a permutation test with 1000 iterations. During each iteration, labels in the training set were permuted and the previously described training process of nested CV and ensembling was re-applied to generate a new set of ensemble beta values per HIF. P-values for individual HIFs were then obtained by comparing beta values in the original ensemble model against the corresponding null distribution of ensemble beta values. Individual HIF P-values were combined into cluster-level P-values via the Empirical Brown's method and corrected using the Benjamini-Hochberg procedure.

### II. EXEMPLARY MODEL ARCHITECTURE FOR HUMAN INTERPRETABLE FEATURES FRAMEWORK

In some embodiments, the deep learning model and/or other models described herein may include a convolutional neural network. For example, a convolutional neural network may be implemented in statistical model 120 (FIG. 1), 420 (FIG. 4). The convolutional neural network may be fully convolutional or may have one or more fully connected layers. In some embodiments, the model may be a different type of neural network model such as, for example, a recurrent neural network, a multi-layer perceptron, and/or a restricted Boltzmann machine. It should be appreciated that the model is not limited to being implemented as a neural network and, in some embodiments, may be a different type of model that may be used to predict annotations for one or more portions of a whole-slide image. For example, the model may be any suitable type of non-linear regression model such as a random forest regression model, a support vector regression model, or an adaptive basis function regression model. As another example, the model may be a Bayesian regression model or any other suitable Bayesian Hierarchical model. In some embodiments, a neural network includes an input layer, an output layer, and one or more hidden layers that define connections from the input layer to the output layer. Each layer may have one or more nodes. For example, the neural network may include at least 5 layers, at least 10 layers, at least 15 layers, at least 20 layers, at least 25 layers, at least 30 layers, at least 40 layers, at least 50 layers, or at least 100 layers. FIG. 11 provides details for training a convolutional neural network in accordance with some embodiments for model predictions of annotations for whole-slide images using the training data.

In some embodiments, the deep learning model can be implemented based on a variety of topologies and/or architectures including deep neural networks with fully connected (dense) layers, Long Short-Term Memory (LSTM) layers, convolutional layers, Temporal Convolutional Layers (TCL), or other suitable type of deep neural network topology and/or architecture. The neural network can have different types of output layers including output layers with logistic sigmoid activation functions, hyperbolic tangent activation functions, linear units, rectified linear units, or other suitable type of nonlinear unit. Likewise, the neural network can be configured to represent the probability distribution over n different classes via, for example, a softmax function or include an output layer that provides a parameterized distribution e.g., mean and variance of a Gaussian distribution.

FIG. 11 schematically shows layers of a convolutional neural network in accordance with some embodiments of the technology described herein. The convolutional neural network may be used to predict annotations for a whole-slide image in accordance with some embodiments of the technology described herein. For example, the convolutional neural network may be used to predict annotations for a whole-slide image. The convolutional neural network may be used because such networks are suitable for analyzing visual images. The convolutional neural network may require no pre-processing of a visual image in order to analyze the visual image. As shown, the convolutional neural network comprises an input layer 704 configured to receive information about the image 702 (e.g., pixel values for all or one or more portions of a whole-slide image), an output layer 708 configured to provide the output (e.g., a classification), and a plurality of hidden layers 706 connected between the input layer 704 and the output layer 708. The plurality of hidden layers 706 include convolution and pooling layers 710 and fully connected layers 712.

The input layer 704 may be followed by one or more convolution and pooling layers 710. A convolutional layer may comprise a set of filters that are spatially smaller (e.g., have a smaller width and/or height) than the input to the convolutional layer (e.g., the image 702). Each of the filters may be convolved with the input to the convolutional layer to produce an activation map (e.g., a 2-dimensional activation map) indicative of the responses of that filter at every spatial position. The convolutional layer may be followed by a pooling layer that down-samples the output of a convolutional layer to reduce its dimensions. The pooling layer may use any of a variety of pooling techniques such as max pooling and/or global average pooling. In some embodiments, the down-sampling may be performed by the convolution layer itself (e.g., without a pooling layer) using striding.

The convolution and pooling layers 710 may be followed by fully connected layers 712. The fully connected layers 712 may comprise one or more layers each with one or more neurons that receives an input from a previous layer (e.g., a convolutional or pooling layer) and provides an output to a subsequent layer (e.g., the output layer 708). The fully connected layers 712 may be described as “dense” because each of the neurons in a given layer may receive an input from each neuron in a previous layer and provide an output to each neuron in a subsequent layer. The fully connected layers 712 may be followed by an output layer 708 that provides the output of the convolutional neural network. The output may be, for example, an indication of which class, from a set of classes, the image 702 (or any portion of the image 702) belongs to. The convolutional neural network may be trained using a stochastic gradient descent type algorithm or another suitable algorithm. The convolutional neural network may continue to be trained until the accuracy on a validation set (e.g., held out images from the training data) saturates or using any other suitable criterion or criteria.

It should be appreciated that the convolutional neural network shown in FIG. 11 is only one example implementation and that other implementations may be employed. For example, one or more layers may be added to or removed from the convolutional neural network shown in FIG. 11. Additional example layers that may be added to the convolutional neural network include: a pad layer, a concatenate layer, and an upscale layer. An upscale layer may be configured to upsample the input to the layer. An ReLU layer may be configured to apply a rectifier (sometimes referred to as a ramp function) as a transfer function to the input. A pad layer may be configured to change the size of the input to the layer by padding one or more dimensions of the input. A concatenate layer may be configured to combine multiple inputs (e.g., combine inputs from multiple layers) into a single output.

As another example, in some embodiments, one or more convolutional, transpose convolutional, pooling, unpooling layers, and/or batch normalization may be included. As yet another example, the architecture may include one or more layers to perform a nonlinear transformation between pairs of adjacent layers. The non-linear transformation may be a rectified linear unit (ReLU) transformation, a sigmoid, and/or any other suitable type of non-linear transformation, as aspects of the technology described herein are not limited in this respect. In some embodiments, any suitable optimization technique may be used for estimating neural network parameters from training data. For example, one or more of the following optimization techniques may be used: stochastic gradient descent (SGD), mini-batch gradient descent, momentum SGD, Nesterov accelerated gradient, Adagrad, Adadelta, RMSprop, Adaptive Moment Estimation (Adam), AdaMax, Nesterov-accelerated Adaptive Moment Estimation (Nadam), AMSGrad.

Convolutional neural networks may be employed to perform any of a variety of functions described herein. For example, a convolutional neural network may be employed to predict tissue or cellular characteristics for a whole-slide image. It should be appreciated that more than one convolutional neural network may be employed to make predictions in some embodiments. For example, a first convolutional neural network may be trained on a set of annotated whole-slide images and a second, different convolutional neural network may be trained on the same set of annotated whole-slide images, but magnified by a particular factor, such as 5×, 10×, 20×, or another suitable factor. The first and second neural networks may comprise a different arrangement of layers and/or be trained using different training data. In some embodiments, the convolutional neural network does not include padding between layers. The layers may be designed such that there is no overflow as pooling or convolution operations are performed. Moreover, layers may be designed to be aligned. For example, if a layer has an input of size N*N, and has a convolution filter of size K, with stride S, then (N−K)/S must be an integer in order to have alignment.

### III. EXEMPLARY IMPLEMENTATIONS AND CLASSIFICATION OF SUBPOPULATIONS AND OTHER GENOMIC FEATURES

Various examples of classifying a patient into subpopulations and validation of such classification are further described. For example, Table 6 lists the breakdown of the molecular subtypes across each indication for cancer types colorectal cancer (CRC) and endometrial (EC).

TABLE 6
The prevalence of each molecular subtype per cancer type

| | MSI | MSS/dMMR | MSS/TMB-H | MSS |
|---|---|---|---|---|
| Colorectal | 64 (14%) | 66 (14%) | 10 (2%) | 324 (70%) |
| cancer | | | | |
| Endometrial | 169 (33%) | 54 (10%) | 41 (8%) | 253 (49%) |
| cancer | | | | |

In CRC, MSI was associated with general immune cell infiltration into the tumor. Both MSI and MSS/dMMR were associated with increased immune cell infiltration, particularly macrophages, into cancer gland lumen tissue. Multivariable HIF models were able to differentiate between MSI and non-MSI tumors (median AUROC: 0.86), and MSS/dMMR from MSS tumors (median AUROC: 0.65), including MSS/TMB-high. In EC, pairwise analysis revealed 61, 34, and 11 HIFs associated with MSI, MSS/dMMR, and MSS/TMB-high when compared to MSS tumors (FDR p<0.05), respectively. Conversely, when comparing MSI, MSS/dMMR, and MSS/TMB-high to each other, no HIFs passed FDR correction.

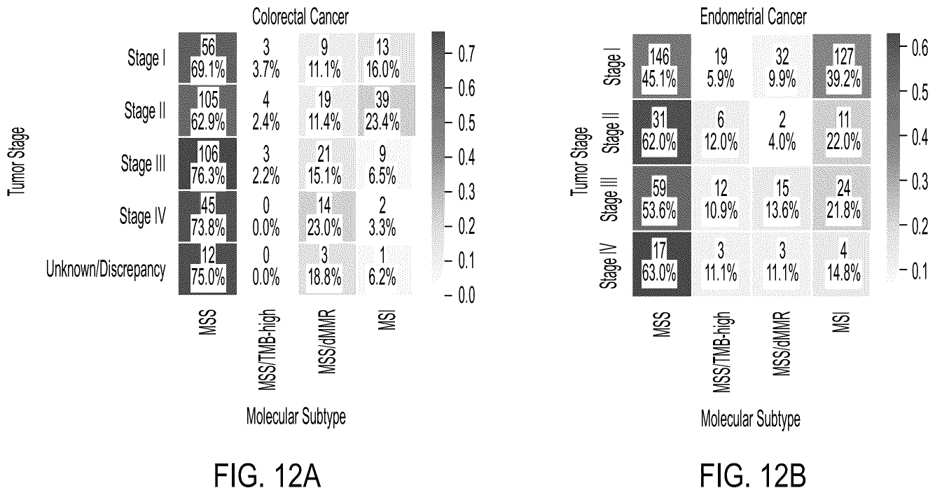

FIG. 12A shows the genomic subpopulations breakdown by tumor stage for colorectal cancer, according to some embodiments. FIG. 12B shows the genomic subpopulations breakdown by tumor stage for endometrial cancer, according to some embodiments, where all samples analyzed were from primary tumors. As shown, the high prevalence of MSI in early-stage tumors tracks with germline alterations in Lynch Syndrome genes, whereas there is increased prevalence of MSS/dMMR as tumors progress to later-stages, suggesting the classification of a patient into subpopulation MSS/dMMR may identify an additional subset of patients without relevant germline alterations that will likely respond to immunotherapy treatment.

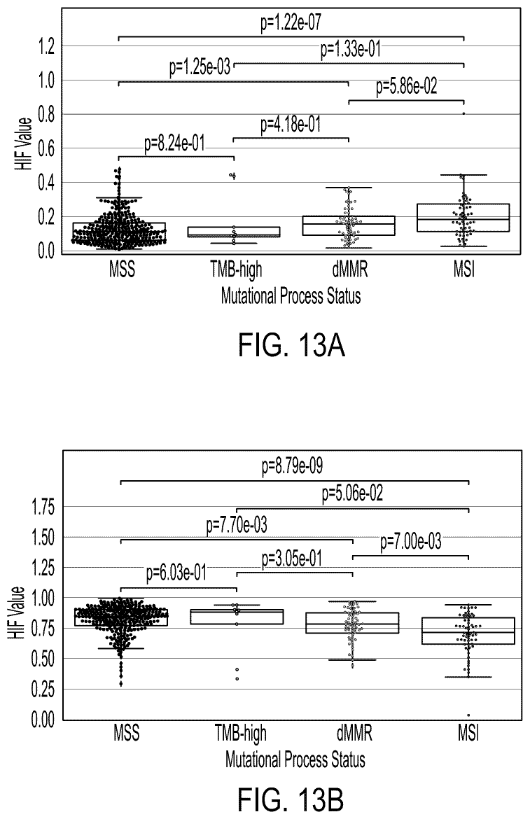

FIG. 13A-13B show the distributions of a positively associated HIF (FIG. 13A) and negatively associated HIF (FIG. 13B) across the four subpopulations for colorectal cancer. As shown, a consistent step-wise pattern is observed for most HIFs that are associated with MSI, suggesting that MSS/dMMR tumors have an intermediate TME phenotype in colorectal cancer.

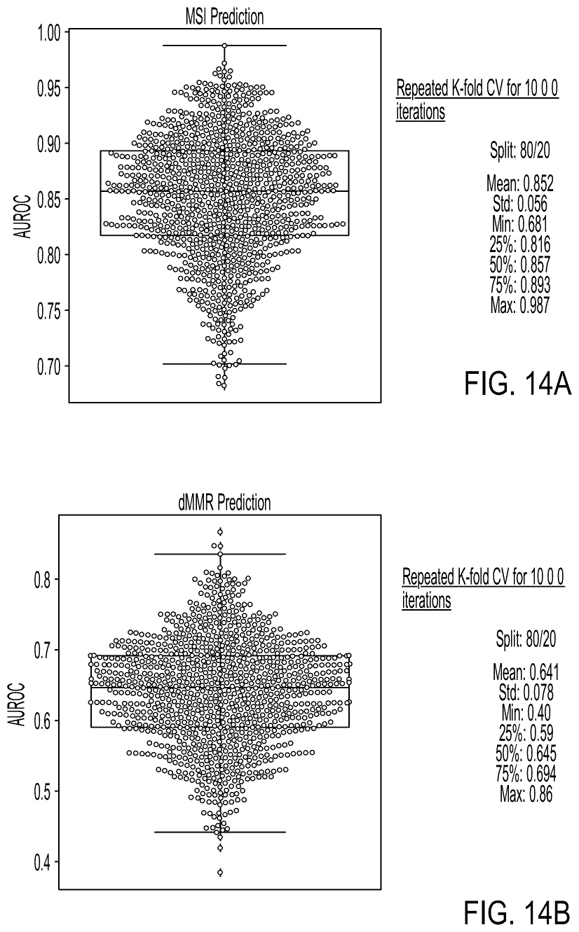

FIG. 14A shows the AUROC curve for MSI prediction in colorectal cancer, and FIG. 14B shows the AUROC curve for dMMR prediction in colorectal cancer, where the predictions were made in the manner as described above using the multivariable HIF models (e.g., FIG. 1). As shown, multivariable HIF models can differentiate MSI tumors from non-MSI tumors, and MSS/dMMR tumors from MSS/TMB-H and MSS tumors in colorectal cancer.

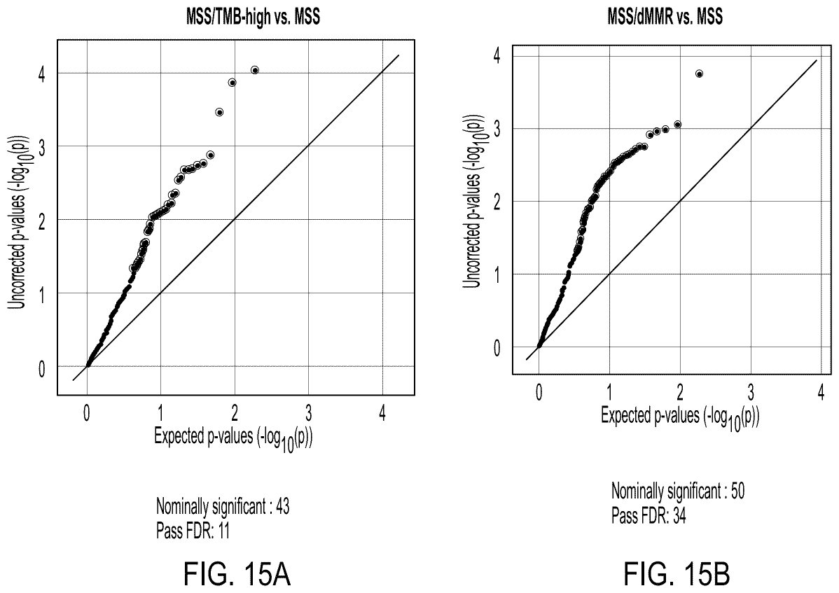

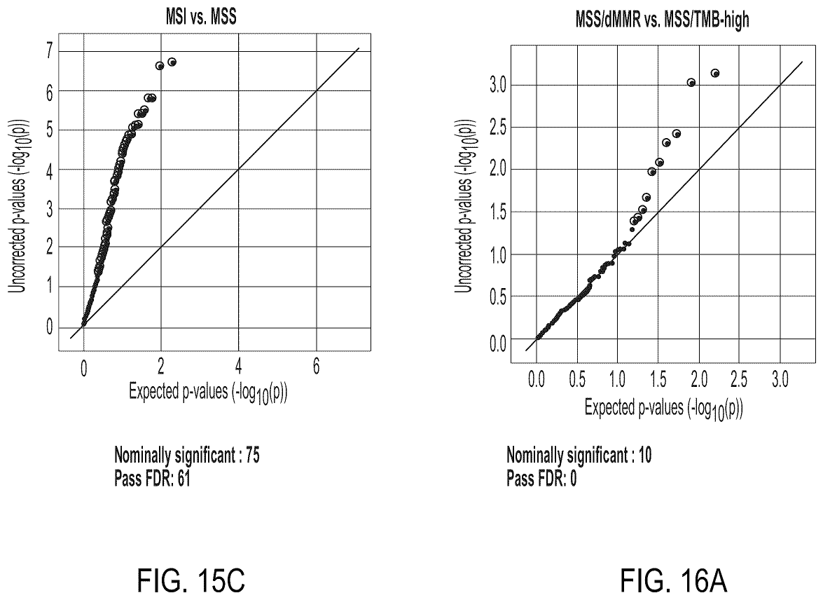

FIG. 15A shows uncorrected p-values between subpopulations MSS/TMB-H and MSS in endometrial cancer; FIG. 15B shows uncorrected p-values between subpopulations MSS/dMMR and MSS in endometrial cancer; and FIG. 15C shows uncorrected p-values between subpopulations MSI and MSS in endometrial cancer, according to some embodiments. As shown, the MSI, MSS/dMMR, and MSS/TMB-H subpopulations are associated with several TME features when compared to MSS tumors in endometrial cancer.

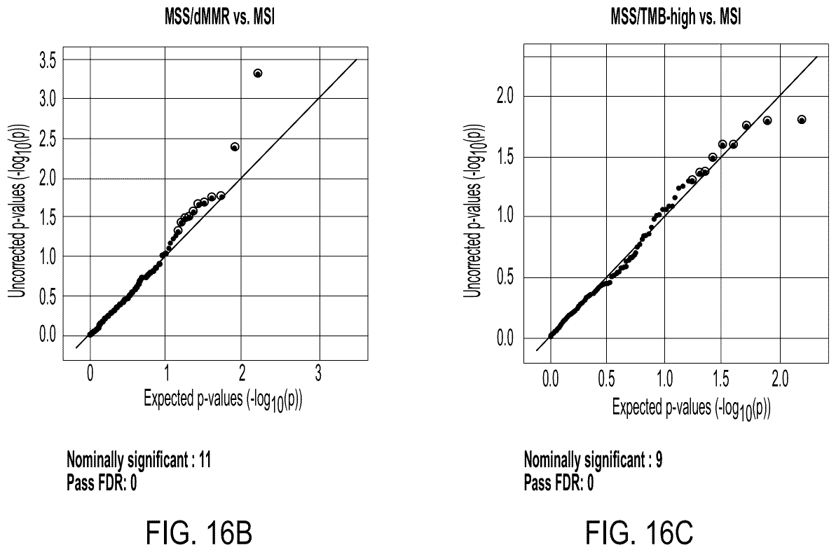

FIG. 16A shows uncorrected p-values between subpopulations MSS/dMMR and MSS/TMB-H in endometrial cancer; FIG. 16B shows uncorrected p-values between subpopulations MSS/dMMR and MSI in endometrial cancer; and FIG. 16C shows uncorrected p-values between subpopulations MSS/TMB-H and MSI in endometrial cancer, according to some embodiments. As shown, in contrast to FIGS. 16A-16C, there is little or no difference between MSI, MSS/dMMR, and MSS/TMB-H tumors in endometrial cancer.

### IV. EXEMPLARY COMPUTER ARCHITECTURE

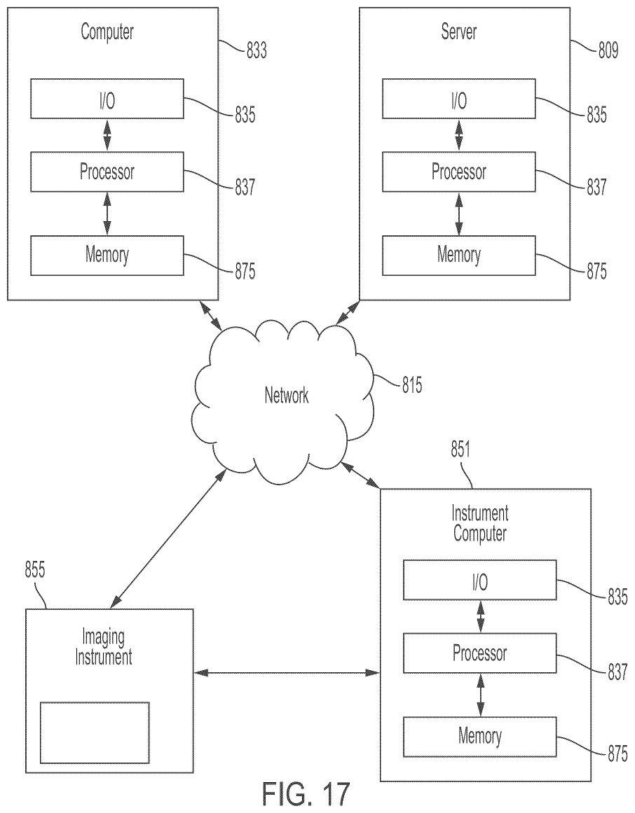

FIG. 17 shows a block diagram of a computer system on which various embodiments of the technology described herein may be practiced. In some embodiments, the system may implement any embodiment described in FIGS. 1-16C. For example, the system may implement processes 100 (FIG. 1), 200 (FIG. 2), 300 (FIG. 3), and 400 (FIG. 4), or any training processes, such as 150 (FIG. 1), 350 (FIG. 3), and 450 (FIG. 4). In some embodiments, the system includes at least one computer 833. Optionally, the system may further include one or more of a server computer 809 and an imaging instrument 855 (e.g., one of the instruments described above), which may be coupled to an instrument computer 851. Each computer in the system includes a processor 837 coupled to a tangible, non-transitory memory device 875 and at least one input/output device 835. Thus the system includes at least one processor 837 coupled to a memory subsystem 875 (e.g., a memory device or collection of memory devices). The components (e.g., computer, server, instrument computer, and imaging instrument) may be in communication over a network 815 that may be wired or wireless and wherein the components may be remotely located or located in close proximity to each other. Using those components, the system is operable to receive or obtain image data such as whole-slide images, pathology images, histology images, or tissue images and annotation and score data as well as test sample images generated by the imaging instrument or otherwise obtained. In certain embodiments, the system uses the memory to store the received data as well as the model data which may be trained and otherwise operated by the processor.

In some embodiments, some or all of the system is implemented in a cloud-based architecture. The cloud-based architecture may offer on-demand access to a shared pool of configurable computing resources (e.g. processors, graphics processors, memory, disk storage, network bandwidth, and other suitable resources). A processor in the cloud-based architecture may be operable to receive or obtain training data such as whole-slide images, pathology images, histology images, or tissue images and annotation and score data as well as test sample images generated by the imaging instrument or otherwise obtained. A memory in the cloud-based architecture may store the received data as well as the model data which may be trained and otherwise operated by the processor. In some embodiments, the cloud-based architecture may provide a graphics processor for training the model in a faster and more efficient manner compared to a conventional processor.

Processor refers to any device or system of devices that performs processing operations. A processor will generally include a chip, such as a single core or multi-core chip (e.g., 12 cores), to provide a central processing unit (CPU). In certain embodiments, a processor may be a graphics processing unit (GPU) such as an Nvidia Tesla K80 graphics card from NVIDIA Corporation (Santa Clara, CA). A processor may be provided by a chip from Intel or AMD. A processor may be any suitable processor such as the microprocessor sold under the trademark XEON E5-2620 v3 by Intel (Santa Clara, CA) or the microprocessor sold under the trademark OPTERON 6200 by AMD (Sunnyvale, CA). Computer systems may include multiple processors including CPUs and or GPUs that may perform different steps of the described methods. The memory subsystem 875 may contain one or any combination of memory devices. A memory device is a mechanical device that stores data or instructions in a machine-readable format. Memory may include one or more sets of instructions (e.g., software) which, when executed by one or more of the processors of the disclosed computers can accomplish some or all of the methods or functions described herein. Each computer may include a non-transitory memory device such as a solid state drive, flash drive, disk drive, hard drive, subscriber identity module (SIM) card, secure digital card (SD card), micro SD card, or solid state drive (SSD), optical and magnetic media, others, or a combination thereof. Using the described components, the system is operable to produce a report and provide the report to a user via an input/output device. An input/output device is a mechanism or system for transferring data into or out of a computer. Exemplary input/output devices include a video display unit (e.g., a liquid crystal display (LCD) or a cathode ray tube (CRT)), a printer, an alphanumeric input device (e.g., a keyboard), a cursor control device (e.g., a mouse), a disk drive unit, a speaker, a touchscreen, an accelerometer, a microphone, a cellular radio frequency antenna, and a network interface device, which can be, for example, a network interface card (NIC), Wi-Fi card, or cellular modem.

### V. ADDITIVE MULTIPLE INSTANCE LEARNING (MIL) MODELS

Described herein is a formulation of Multiple Instance Learning (MIL) models that enables interpretability while maintaining similar predictive performance. MIL models of the types described herein may be used to predict, and identify whole slide image (WSI)-derived features associated with MSI positivity status and evidence of dMMR in microsatellite stable (MSS) tumors. The models developed by the inventors and described herein enable spatial credit assignment such that the contribution of each region in an image can be accurately computed and visualized. The resulting spatial credit assignment coincides with regions used by pathologists during diagnosis and improves upon classical attention heatmaps from attention MIL models. These models can debug model failures, identify spurious features, and highlight class-wise regions of interest, enabling their use in high-stakes environments such as clinical decision-making.

Histopathology is the study and diagnosis of disease by microscopic inspection of tissue. Histologic examination of tissue samples plays a key role in both clinical diagnosis and drug development. It is regarded as medicine's ground truth for various diseases and is important in evaluating disease severity, measuring treatment effects, and biomarker scoring. A differentiating feature of digitized tissue slides or whole slide images (WSI) is their extremely large size, often billions of pixels per image. In addition to being large, WSIs are extremely information dense, with each image containing thousands of cells and detailed tissue regions that make manual analysis of these images challenging. This information richness makes pathology an excellent application for machine learning, and indeed there has been tremendous progress in recent years in applying machine learning to pathology data.

One of the most important applications of machine learning in digital pathology involves predicting patient's clinical characteristics from a WSI image. Models need to be able to make predictions about the entire slide involving all the patient tissue available; these predictions are referred to as “slide-level”. To overcome the challenges presented by the large size of these images, previous methods have used smaller hand engineered representations, built from biological primitives in tissue such as cellular composition and structures. Another common way to overcome the challenges presented by the size of WSIs is to break the slide into thousands of small patches, train a model with these patches to predict the slide-label, and then use a secondary model to learn an aggregation function from patch representations to slide-level label. Both methods are not trained in an end-to-end manner and suffer from sub-optimal performance. The second method also suffers from an incorrect assumption that each patch from a slide has the same label as the overall slide.

MIL is a weakly supervised learning technique which attempts to learn a mapping from a set of instances (called a bag) to a single label associated with the whole bag. MIL can be applied to pathology by treating patches from slides as instances which form a bag and a slide-level label is associated with each bag to learn a bag predictor. This circumvents the need to collect patch-level labels and allows end-to-end training from a WSI. The MIL assumption that at least one patch among the set of patches is associated with the target label works well for many biological problems. For example, the MIL assumption holds for the task of cancer diagnosis; a sufficiently large bag of instances or patches from a cancerous slide will contain at least one cancerous patch whereas a benign slide will never contain a cancerous patch. In recent years, attention-based pooling of patches has been shown to be successful for MIL problems. Using neural networks with attention MIL has become the standard for end-to-end pathology models as it provides a powerful, yet efficient gradient based method to learn a slide-to-label mapping. In addition to superior performance, these models encode some level of spatial interpretability within the model through visualization of highly attended regions.

The sensitive nature of the medical imaging domain requires deployed machine learning models to be interpretable for multiple reasons. First, it is critical that models do not learn spurious shortcuts over true signal and can be debugged if such failure modes exist. Interpretability and explainability methods have been shown to help identify some of these data and model deficiencies. Secondly, for algorithms in medical decision-making, accountability and rigorous validation precedes adoption. Interpretable models can be easier to validate and thus build trust. Specifically, users can verify that model predictions are generated using biologically concordant features that are supported by scientific evidence and are similar to the those identified by human experts. Thirdly, use-cases involving a human expert such as decision-support require the algorithm to give a visual cue which highlights the regions to be examined more carefully. In these applications, a predicted score is insufficient and needs to be complemented with a highlighted visual region associated with the model's prediction. For machine learning models in pathology, spatial credit assignment can be defined as attributing model predictions to specific spatial regions in the slide. Various post-hoc interpretability techniques like gradient based methods and Local Interpretable Model-agnostic Explanation (LIME) have been used to this end. However, gradient based methods which try to construct model-dependent saliency maps are often insensitive to the model or the data. This makes these post-hoc methods unreliable for spatial attribution as they provide poor localization and do not reflect the model's predictions.

Model-agnostic methods like Shapley values or LIME involve intractable computations for large image data and thus need approximations like locally fitting explanations to model predictions, which can lead to incorrect attribution. Applying attention MIL in weakly supervised problems in pathology leads to learning of the attention scores for each patch. These scores can be used as a proxy for patch importance, thus helping in spatial credit assignment. This way of interpreting MIL models has been used commonly in the literature to create spatial heatmaps, image overlays that indicate credit assignment, for free without applying any post-hoc technique. The attention values that scale patch feature representations have a non-linear relationship to the final prediction, making their visual interpretation inexact and incomplete.

To address these issues, the inventors propose a formulation of MIL which induces intrinsically interpretable heatmaps. This model is referred to herein as “additive MIL.” It allows for precise decomposition of a model prediction in terms of spatial regions of the input. These models, instead of being applied to arbitrary features, are grounded as patch instances in the MIL formulation which allows precise (e.g., exact) credit assignment for each patch in a bag. Specifically, this is achieved by constraining the space of predictor functions (the classification or regression head at the final layer) in the MIL setup to be additive in terms of instances. Therefore, the contribution of each patch or instance in a bag can be traced back from the final predictions. These additive scores reflect the true marginal contribution of each patch to a prediction and can be visualized as a heatmap on a slide for various applications like model debugging, validating model performance, and identifying spurious features.

The inventors have recognized and appreciated that these benefits can be achieved without any material loss of predictive performance even though the predictor function is constrained to be additive. This represents a substantial improvement over previous MIL implementations.

An attention MIL model can be seen as a 3-part model involving:
a featurizer (f), typically a deep convolutional neural network (CNN),
an attention module (m), which induces a soft attention over N patches and is used to scale each patch feature, and
a predictor (p), which takes the attended patch representations, aggregates them using a permutation invariant function like sum pooling over the N patches, and then outputs a prediction. This MIL model g (x) is given by:

$$g ( x ) = ( p ∘ m ∘ f ) ( x ) \qquad ( 1 )$$

$${m}_{i} ( x ) = {α}_{i} f ( {x}_{i} ) where {α}_{i} = {softmax}_{i} ( {ψ}_{m} ( x ) ) \qquad ( 2 )$$

$$p ( x ) = {ψ}_{p} {\sum }_{i = 1}^{N} ( {m}_{i} ( x ) ) \qquad ( 3 )$$

where ψₘ and ψₚ are multilayer perceptrons (MLPs) with non-linear activation functions. The attention scores αᵢ learned by the model can be treated as patch importance scores and are used to interpret MIL models.

The inventors have recognized and appreciated that there are several limitations in doing spatial attribution using these attention scores. For example, consider the task of classifying a slide into benign, suspicious or malignant.

First, since the attention weights are used to scale the patch features used for the prediction task, a high attention weight only means that the patch might be needed for the prediction downstream. Therefore, a high attention score for a patch can be a necessary but not sufficient condition for attributing a prediction to that patch. Similarly, patches with low attention can be important for the downstream prediction since the attention scores are related non-linearly to the final classification or regression layer. For example, in a malignant slide, non-tumor regions might get highlighted by the attention scores since they need to be represented at the final classification layer to provide discriminative signal. However, this does not imply malignant prediction should be attributed to non-malignant regions, nor that these regions would be useful to guide a human expert.

Second, the contribution of a patch to the final prediction can be either positive (excitatory) or negative (inhibitory), however attention scores do not distinguish between the two. A patch might be providing strong negative evidence for a class but will be highlighted in the same way as a positive patch. For example, benign mimics of cancer are regions which visually look like cancer but are normal benign tissue. These regions are useful for the model to provide negative evidence for the presence of cancer and thus might have high attention scores. While attending to these regions may be useful to the model, they may complicate human interpretation of resulting heatmaps.

Third, attention scores do not provide meaningful information about the class-wise importance of a patch, but only that a patch was weighted by a certain magnitude for generating the prediction. In the case of multiclass classification, this becomes problematic as a high attention score on a patch can mean that it might be useful for any of the multiple classes. Different regions in the slide might be contributing to different classes which are indistinguishable in an attention heatmap. For example, if a patch has high attention weight for benign-suspicious-malignant classification, it can be interpreted as being important for any one or more of the classes. This makes the attention scores ineffective for verifying the role of individual patches for a slide-level prediction.

Fourth, using attention scores to assess patch contribution ignores patch interactions at the classification stage. For example, two different tumor patches might have moderate attention scores, but when taken together in the final classification layer, they might jointly provide strong and sufficient information for the slide being malignant. Thus, computing marginal patch contributions for a bag needs to be done at the classification layer and not the attention layer since attention scores do not capture patch interactions and thus can underestimate or overestimate contributions to the final prediction.

These limitations in interpreting attention MIL heatmaps motivate the formulation of a traceable predictor function, where model predictions can be specified in terms of patch contributions (both positive and negative) for each class. The inventors have developed additive MIL models to address the aforementioned limitations. The inventors have recognized and appreciated that it is desirable that the approaches described herein be intrinsic to the model, as opposed to being post-hoc approaches. This prevents incorrect assumptions about the model without the need for post-hoc modeling. It also prevents many pitfalls of traditional saliency methods.

The inventors have further recognized and appreciated that it is desirable that attribution be performed in terms of instances only. For pathology, this means that the prediction should be attributed to individual patches. This constraint enables expression of bag predictions in terms of marginal instance contributions.

The inventors have further recognized and appreciated that it is desirable that the model be able to distinguish between excitatory and inhibitory patch contributions. Some models provide per-class contributions for classification problems. To enable the desired instance-level credit assignment in MIL, according to some embodiments, the final predictor is re-framed to be an additive function of individual instances. This can be expressed in accordance with the following example expression:

$${p}_{Additive} ( x ) = {\sum }_{i = 1}^{N} {ψ}_{p} ( {m}_{i} ( x ) ) \qquad ( 4 )$$

Making this change results in the final predictor only being able to implement patch-additive functions on top of arbitrarily complex patch representations. This provides both complexity of the learned representations as well as a traceable patch contribution for a given prediction, which solves the spatial credit assignment problem. The function ψₚ(mᵢ(x)) is the class-wise contribution for patch i in the bag. At inference, ψ produces a R^{C}^{ₓ}^{N} for a classification problem where C is the number of classes and N is the number of patches in a bag. Thus, a class-wise score for each patch is obtained, which when summed gives the final logits for the prediction problem. These scores can be visualized by constructing a heatmap from the visual representation of patch-wise contributions for each class. The sign of the patch contribution determines whether the patch is excitatory or inhibitory towards each class since positive values add to the final logit while negative values bring down the final class logit.

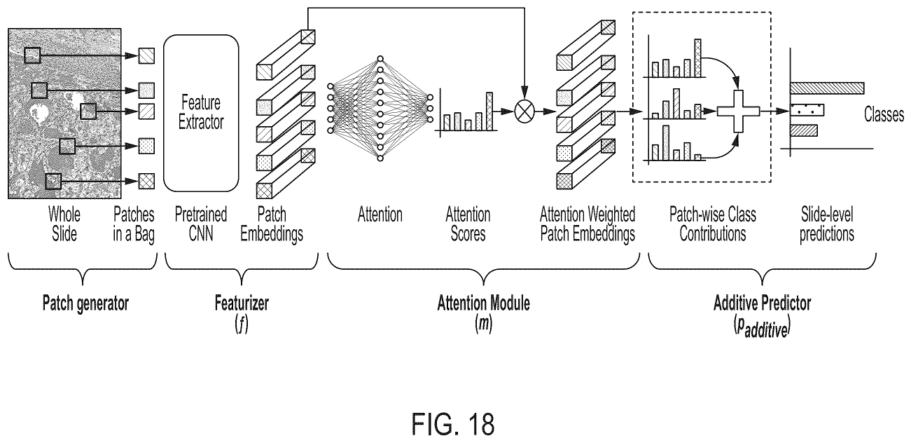

FIG. 18 illustrates an example of an additive MIL model, in accordance with some embodiments. The model includes a patch generator, a featurizer (f), an attention module (m) and an additive predictor (p_{additive}). The patch generator is configured to generate a bag with a plurality of patches from an input image. Each patch includes a distinct portion of the input image. The featurizer includes a neural network (e.g., convolutional) model configured to generate a plurality of patch embeddings using at least a portion of the bag. The attention module is configured to determine an attention score for each of the plurality of patch embeddings. Further, the attention module generates a plurality of attention weighted patch embeddings by scaling the plurality of patch embeddings using the attention scores. The additive predictor is configured to aggregate the plurality of attention weighted patch embeddings to generate a plurality of patch-wise class contributions. Each patch-wise class contribution represents a contribution of a corresponding class. Further, the additive predictor computes a plurality of predictions from the patch-wise class contributions using an additive function. Optionally, a heatmap of the image may be generated. The heatmap may identifying patch-wise class contributions associated with each class, as described in detail further below.

It should be noted that a convolutional neural network is used as an example of a model that may be used in accordance with some embodiments. However, it should be appreciated that other types of statistical models may alternatively be used, and embodiments are not limited in this respect. Other types of statistical models that may be used include a support vector machine, a neural network, a regression model, a random forest, a clustering model, a Bayesian network, reinforcement learning, metric learning, a genetic algorithm, or another suitable statistical model.

Additive MIL models provide precise (e.g., exact) patch contribution scores which are additively related to the prediction. This additive coupling of the model and the interpretability method makes the spatial scores precisely mirror the invariances and the sensitivities of the model, thus making them intrinsically interpretable.

Additive MIL models allow decomposing the patch contributions and attributing them to individual classes in a classification problem. This allows not only to assign the prediction to a region, but also to determine to which class it contributes. This is helpful in cases where signal for multiple classes exist within the same slide.

Additive MIL models allow for both positive and negative contributions from a patch. This can help distinguish between areas which are important because they provide evidence for the prediction and those which provide evidence against.

### VI. CONCLUSION

Various embodiments described in FIGS. 1-18 provide advantages in improved patient outcomes through identifying patients that may respond to immunotherapy treatment, and thus administering proper treatment to the patients. Drivers of immunotherapy response is key to improving patient outcomes. Molecular features, which are cancer cell intrinsic, are typically considered. The technologies described herein show that extrinsic factors like tumor microenvironment composition, may enable identification of more patients amenable to immunotherapy. Specifically, digital pathology-derived features associated with MSI status can be identified, where these features are shown to be present in some MSS patients, with and without dMMR, that will likely benefit from immunotherapy.

It is to be appreciated that embodiments of the methods and apparatuses discussed herein are not limited in application to the details of construction and the arrangement of components set forth in the following description or illustrated in the accompanying drawings. The methods and apparatuses are capable of implementation in other embodiments and of being practiced or of being carried out in various ways. Examples of specific implementations are provided herein for illustrative purposes only and are not intended to be limiting. In particular, acts, elements and features discussed in connection with any one or more embodiments are not intended to be excluded from a similar role in any other embodiments.

Also, the phraseology and terminology used herein is for the purpose of description and should not be regarded as limiting. Any references to embodiments or elements or acts of the systems and methods herein referred to in the singular may also embrace embodiments including a plurality of these elements, and any references in plural to any embodiment or element or act herein may also embrace embodiments including only a single element. References in the singular or plural form are not intended to limit the presently disclosed systems or methods, their components, acts, or elements.

Also, various inventive concepts may be embodied as one or more processes, of which examples have been provided. The acts performed as part of each process may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, or ordinary meanings of the defined terms.

The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms. Any references to front and back, left and right, top and bottom, upper and lower, and vertical and horizontal are intended for convenience of description, not to limit the present systems and methods or their components to any one positional or spatial orientation.

As referred to herein, the term “in response to” may refer to initiated as a result of or caused by. In a first example, a first action being performed in response to a second action may include interstitial steps between the first action and the second action. In a second example, a first action being performed in response to a second action may not include interstitial steps between the first action and the second action.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In this application, unless otherwise clear from context, (i) the term “a” means “one or more”; (ii) the term “or” is used to mean “and/or” unless explicitly indicated to refer to alternatives only or the alternative are mutually exclusive, although the disclosure supports a definition that refers to only alternatives and “and/or”; (iii) the terms “comprising” and “including” are understood to encompass itemized components or steps whether presented by themselves or together with one or more additional components or steps; and (iv) where ranges are provided, endpoints are included.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

Having thus described several aspects of at least one embodiment, it is to be appreciated that various alterations, modifications, and improvements will readily occur to those skilled in the art. Such alterations, modifications, and improvements are intended to be part of this disclosure, and are intended to be within the spirit and scope of the systems and methods described herein. Accordingly, the foregoing description and drawings are by way of example only.

What is claimed is:

1. A method for classifying a patient into one or more subpopulations of a plurality of subpopulations associated with solid tumor based on human interpretable image features extracted from a pathology image of the patient, the method comprising, by one or more processors:
using a first statistical model to determine one or more cell-type labels and/or one or more tissue-type segmentations associated with a pathology image of a patient;
determining a plurality of human interpretable image features based on the one or more cell-type labels and/or the one or more tissue-type segmentations associated with the pathology image; and
using a second statistical model to classify the patient into one or more subpopulations of a plurality of subpopulations associated with solid tumor based on the plurality of human interpretable image features, the plurality of subpopulations comprising at least a first subpopulation having MSS/dMMR.

2. The method of claim 1, wherein the pathology image comprises a whole-slide image.

3. The method of claim 1, further comprising predicting whether the patient will respond to immunotherapy treatment for the solid tumor, the predicting comprising:
in response to classifying the patient into the first subpopulation having MSS/dMMR, predicting that the patient will respond to the immunotherapy treatment.

4. The method of claim 3, wherein the plurality of subpopulations further comprise a second subpopulation having MSI, a third subpopulation having MSS/TMB-high in the pathology image, and a fourth subpopulation that does not have MSI, dMMR and TMB-high.

5. The method of claim 4, wherein predicting whether the patient will respond to immunotherapy treatment for the solid tumor further comprises:
predicting that the patient will respond to the immunotherapy treatment in response to classifying the patient into the second subpopulation having MSI, or the third subpopulation having MSS/TMB-high; otherwise
predicting that the patient will not respond to the immunotherapy treatment.

6. The method of claim 5, wherein classifying the patient into the one or more subpopulations of the plurality of subpopulations associated with solid tumor comprises:
using a first portion of the second statistical model to predict whether the patient has MSI or MSS, the first portion of the second statistical model is associated with a first subset of the plurality of human interpretable image features; and
in response to predicting that the patient has MSI, classifying the patient into the second subpopulation having MSI.

7. The method of claim 6, wherein classifying the patient into the one or more subpopulations of the plurality of subpopulations associated with solid tumor further comprises:
in response to predicting that the patient does not have MSI, using a second portion of the second statistical model to predict whether the patient has dMMR, the second portion of the second statistical model is associated with a second subset of the plurality of human interpretable image features; and
in response to predicting that the patient has dMMR, classifying the patient into the first subpopulation having MSS/dMMR.

8. The method of claim 7, wherein classifying the patient into the one or more subpopulations of the plurality of subpopulations associated with solid tumor further comprises:
in response to predicting that the patient does not have MSI, using a third portion of the second statistical model to predict whether the patient has TMB-high, the second portion of the second statistical model is associated with a third subset of the plurality of human interpretable image features; and
in response to predicting that the patient has TMB-high, classifying the patient into the third subpopulation having MSS/TMB-high.

9. The method of claim 1, wherein the first statistical model comprises a convolutional neural network.

10. The method of claim 9, wherein the second statistical model comprises a logistic regression model.

11. The method of claim 1, wherein:
the one or more cell-type labels comprise at least one selected from the group consisting of cancer epithelium, cancer stroma, cancer gland lumen, buds and poorly differentiated clusters, mucin, and necrosis; and
the one or more tissue-type segmentations comprise at least one selected from the group consisting of cancer cell, fibroblast, macrophage, lymphocyte, and plasma cell.

12. The method of claim 1, wherein:
the one or more cell-type labels comprise at least one selected from the group consisting of cancer epithelium, cancer stroma, necrosis, and normal cell-type; and
the one or more tissue-type segmentations comprise at least one selected from the group consisting of cancer cell, fibroblast, macrophage, lymphocyte, plasma cell, endothelial cell, neutrophil, and eosinophil.

13. The method of claim 1, wherein:
the one or more cell-type labels comprise at least one selected from the group consisting of Cancer epithelium, cancer stroma, mucin, and necrosis; and
the one or more tissue-type segmentations comprise at least one selected from the group consisting of Cancer cell, fibroblast, macrophage, lymphocyte, plasma cell, neutrophil, or eosinophil.

14. The method of claim 1, wherein the second statistical model comprises associations between human interpretable image features and the plurality of subpopulations.

15. The method of claim 14, wherein the second statistical model is trained over a plurality of training pathology images, the training configured to:
receive a plurality of training pathology images belonging to known molecular subpopulations;
use the first statistical model to determine cell and/or tissue characteristics from the plurality of training pathology images;
determine one or more human interpretable image features based on the cell and/or tissue characteristics of the plurality of training pathology images; and
identify a pairwise association between the one or more human interpretable image features and a subpopulation of the plurality of subpopulations.

16. The method of claim 15, wherein the identifying the pairwise association is performed using a Mann-Whitney test.

17. The method of claim 1, wherein the solid tumor comprises at least one selected from the group consisting of colorectal cancer, endometrial cancer, and gastric cancer.

18. A method for classifying a patient into one or more subpopulations of a plurality of subpopulations associated with solid tumor from a pathology image of the patient, the method comprising, by one or more processors:
receiving one or more pathology images of a patient;
using a statistical model and the one or more pathology images as input to the statistical model to classify the patient into one or more subpopulations of a plurality of subpopulations associated with solid tumor, the plurality of subpopulations comprising at least a first subpopulation having MSS/dMMR; and
in response to classification of the patient, predicting whether the patient will respond to immunotherapy treatment for the solid tumor, the predicting comprising:
in response to classifying the patient into the first subpopulation having MSS/dMMR, predicting that the patient will respond to the immunotherapy treatment.

19. The method of claim 18, wherein the one or more pathology images of the patient each comprise a whole-slide image.

20. The method of claim 18, wherein the plurality of subpopulations further comprise a second subpopulation having MSI, a third subpopulation having MSS/TMB-high in the pathology image, and a fourth subpopulation that does not have MSI, dMMR and TMB-high.

21. The method of claim 20, wherein predicting whether the patient will respond to the immunotherapy treatment for the solid tumor further comprises:
predicting that the patient will respond to the immunotherapy treatment in response to classifying the patient into the second subpopulation having MSI, or the third subpopulation having MSS/TMB-high; otherwise
predicting that the patient will not respond to the immunotherapy treatment.

22. The method of claim 18, wherein the solid tumor comprises one or more of colorectal cancer, endometrial cancer, or gastric cancer.

23. A method for predicting whether a patient will respond to immunotherapy treatment associated with solid tumor based on human interpretable image features extracted from a pathology image of the patient, the method comprising, by one or more processors:
using a first statistical model to determine one or more cell-type labels and/or one or more tissue-type segmentations associated with a pathology image of a patient;
determining a plurality of human interpretable image features based on the one or more cell-type labels and/or the one or more tissue-type segmentations associated with the pathology image; and
using a second statistical model and the plurality of human interpretable image features as input to the second statistical model to predict whether the patient will respond to immunotherapy treatment for the solid tumor.

24. The method of claim 23, wherein the solid tumor comprises one or more of colorectal cancer, endometrial cancer, or gastric cancer.
the predicting comprising:
in response to classifying the patient into a first subpopulation having MSS/dMMR, predicting that the patient will respond to the immunotherapy treatment.

25. The method of claim 23, wherein the plurality of subpopulations further comprise a second subpopulation having MSI, a third subpopulation having MSS/TMB-high in the pathology image, and a fourth subpopulation that does not have MSI, dMMR and TMB-high.

26. The method of claim 25, wherein predicting whether the patient will respond to immunotherapy treatment for the solid tumor further comprises:
predicting that the patient will respond to the immunotherapy treatment in response to classifying the patient into a second subpopulation having MSI, or a third subpopulation having MSS/TMB-high; otherwise
predicting that the patient will not respond to the immunotherapy treatment.
